(19) United States
(12) Patent Application Publication
(10) Pub. No.: US 2025/0064741 A1
(43) Pub. Date: Feb. 27, 2025

(54) METHODS FOR ENHANCED NUCLEIC ACID DELIVERY

(71) Applicant: CORNELL UNIVERSITY, Ithaca, NY (US)

(72) Inventors: Zhefan YUAN, Ithaca, NY (US); Sijin LUOZHONG, Ithaca, NY (US); Shaoyi JIANG, Ithaca, NY (US)

(73) Assignee: CORNELL UNIVERSITY, Ithaca, NY (US)

(21) Appl. No.: 18/722,792

(22) PCT Filed: Dec. 20, 2022
(86) PCT No.: PCT/US2022/053465

§ 371 Date: Jun. 21, 2024

Related U.S. Application Data
(60) Provisional application No. 63/292,302, filed on Dec. 21, 2021.

(51) Int. Cl. A61K 9/127; A61K 9/51; A61K 31/663; A61K 31/675; A61K 31/7105; C07K 16/28

(52) U.S. Cl. CPC A61K 9/1272; A61K 9/5123; A61K 31/663; A61K 31/675; A61K 31/7105; C07K 16/2866

(57) ABSTRACT

A method for delivery of a nucleic acid. comprising administering a lipid nanoparticle composition loaded with the nucleic acid to a subject in which monocytes and/or macrophages have been depleted. thereby delivering the nucleic acid into the subject, the method may more particularly be practiced according to the following steps: a) depleting monocytes and/or macrophages in a subject: and b) administering a lipid nanoparticle composition loaded with the nucleic acid to the subject, thereby delivering the nucleic acid into the subject.

### CROSS REFERENCE TO RELATED APPLICATIONS

This application claims the benefit of priority from U.S. Provisional Application No. 63/292,302, filed on Dec. 21, 2022, which is herein incorporated by reference in its entirety.

### FIELD OF THE DISCLOSURE

The present disclosure generally relates to methods of enhanced delivery of nuclei acids, such as mRNA, using lipid nanoparticle (LNP) formulations.

### BACKGROUND

In vivo delivery of nucleic acids, such as DNA, siRNA and mRNA, holds the potential to revolutionize vaccination, enzyme replacement therapies, and the treatment of genetic disorders. Compared to conventional protein therapeutics, mRNA therapeutics, in particular, have shown several advantages, including rapid and scalable production. The most prominent example is Moderna and Pfizer/BioNTech mRNA vaccines, which have been used worldwide to combat the coronavirus disease 2019 (COVID-19) pandemics. Beyond vaccines against infectious diseases, mRNA therapeutics have shown promising applications in other fields, such as enzyme replacement therapies and gene correction against genetic disorders.

Successful implementation of mRNA therapeutics is greatly attributed to the development of the lipid nanoparticle (LNP) that protects mRNA from degradation/clearance and assists in cellular uptake and mRNA endosome escape. A typical LNP formulation is made up of ionizable cationic lipids, helper lipids, cholesterol, and polyethylene glycol (PEG)-lipids, forming a particle around 100 nm in diameter. Nanoparticle association with the host highly evolved mononuclear phagocytic system (MPS) is a function of particle opsonization upon contact with blood and rapid recognition of these opsonins via the MPS. This is particularly observed in structurally distinct fenestrated vasculature via liver Kupffer cells and splenic macrophages. If these macrophagic cells are indeed responsible for high particle clearance rates, disappointing imaging and therapeutic efficacy due to poor delivery efficiencies to specific targets and increased clearance organ accumulation are anticipated. Thus, there would be a significant benefit in a method that could employ LNPs while circumventing the scavenging and breaking down of the LNPs by phagocytes; however, such a method has thus far remained elusive.

### SUMMARY

The present disclosure is foremost directed to methods for improving the efficacy of nucleic acid (e.g., mRNA) therapeutics delivered by lipid nanoparticles (LNPs). The nucleic acid being delivered may be, for example, messenger RNA (mRNA), small interfering RNA (siRNA), microRNA (miRNA), circularRNA (circRNA), long-noncoding RNA (IncRNA), antisense oligonucleotide (ASO), CRISPR-related RNA, Cas nuclease mRNA, guide RNA, or single-guide RNA. The method enhances the efficiency of mRNA-LNPs delivery in a subject by pre-depletion of monocytes/macrophages in the subject prior to the administration of mRNA-LNPs. The LNP compositions described herein advantageously possess low immunogenicity, long circulation capabilities, and targeting ability. The LNP compositions can also be functionalized with a targeting agent (e.g., ligand) to bind to specific cells or cellular components to enhance the nucleic acid delivery. In some embodiments, the targeting ligand is or includes a phosphoserine (PS) moiety. Thus, the method described herein can be useful in a range of biotherapeutics, such as vaccines, for treating or preventing infectious diseases, cancers, and improving a subject's health or well-being. By temporary depletion of phagocytes, more LNPs could accumulate in desirable tissues and targeted cells, thus resulting in enhanced mRNA delivery or maintained efficacy with lowered dose. Without the competitive uptake by macrophages, the LNP could mainly accumulate in targeted tissues and cells. This methodology is beneficial in protein replacement or gene editing therapies for rare diseases and resulting enhanced mRNA delivery or maintained efficacy with lowered dose.

More specifically, the methods disclosed herein are directed to delivery of a nucleic acid into a subject in which monocytes and/or macrophages have been depleted. The method comprises administering a lipid nanoparticle composition loaded with a nucleic acid to the subject in which monocytes and/or macrophages have been depleted. In some embodiments, the method includes the following steps: a) depleting monocytes and/or macrophages in the subject; and b) administering a lipid nanoparticle composition loaded with a nucleic acid to the subject, thereby delivering the nucleic acid into the subject. By temporarily depleting phagocytes, more LNPs are permitted to accumulate in desirable tissues or targeted cells, thereby resulting in enhanced nucleic acid (e.g., mRNA) delivery or maintained efficacy with lowered dose. In some embodiments, depleting monocytes and/or macrophages is accomplished by administering small molecule drugs. In other embodiments, depleting monocytes and/or macrophages is accomplished by administering liposome compositions loaded with small molecule drugs. In other embodiments, depleting monocytes and/or macrophages is accomplished by administering antibodies configured to deplete monocytes and/or macrophages, wherein the antibodies include, but are not limited to anti-CD115, anti-CCR2, anti-Ly6C, anti-Gr-1. In other embodiments, depleting monocytes and/or macrophages is accomplished by administering one or more of liposome clondronate (LipoD) and liposome zolendrate. Using any of the above methods, the depletion of monocytes and/or macrophages may be accomplished within hours, days, or weeks.

The end result of the present methods is an enhanced delivery of the nucleic acid into the subject, which is typically a mammal, more particularly a human subject. In some embodiments, the nucleic acid encodes a protein that is expressed in the subject. In other embodiments, the nucleic acid is a component of a gene editing machinery. In other embodiments, the nucleic acid is delivered into one or more of liver, brain, spleen, lymph nodes, kidneys, and lungs. In some embodiments, the enhanced delivery of the nucleic acid results in improved protein expression of protein encoded by the delivered nucleic acid. In other embodiments, the enhanced delivery of nucleic acid results in improved gene editing efficiency by the delivered nucleic acid. In other embodiments, the method provides an enhanced nucleic acid delivery into one or more of the liver, brain, spleen, lymph nodes, kidneys, and lungs.

In some embodiments, the LNP composition includes: (i) at least one zwitterionic polymer-containing lipid in which a lipid moiety is covalently attached to a zwitterionic polymer; (ii) at least one non-cationic lipid selected from charged and uncharged lipids, wherein the non-cationic lipid is not attached to a polymer; (iii) at least one cationic or ionizable lipid containing a secondary, tertiary, or quaternary amino group and (iv) at least one nucleic acid, such as mRNA. In some embodiments, the lipid moiety in component (i) is a diacylglyceride. In some embodiments, component (i) excludes a polyalkylene oxide segment. In some embodiments, the zwitterionic polymer in component (i) is a poly(carboxybetaine) (PCB), a poly(sulfobetaine), a poly(phosphobetaine), poly(phosphatidylcholine), glutamic acid-lysine (EK)-containing polypeptide, a poly(trimethylamine N-oxide) polymer, or a poly(zwitterionic phosphatidyl serine). In some embodiments, the zwitterionic polymer in component (i) is a betaine polymer, such as a poly(carboxybetaine), poly (sulfobetaine), or poly (phosphobetaine) polymer. In some embodiments, the non-cationic lipid in component (ii) contains a zwitterionic moiety, wherein the zwitterionic moiety may be selected from phosphobetaine, phosphatidylcholine, carboxybetaine, sulfobetaine, trimethylamine N-oxide, glutamic acid-lysine (EK)-containing peptide, or zwitterionic phosphatidyl serine moiety. In some embodiments, the non-cationic lipid is selected from dioleoylphosphatidylglycerol (DOPG), dipalmitoylphosphatidylglycerol (DPPG), dioleoylphosphatidylethanolamine (DOPE), palmitoyloleoyl-phosphatidylethanolamine (POPE), dipalmitoylphosphatidylethanolamine (DPPE), dimyristoylphosphoethanolamine (DMPE), distearoylphosphatidylethanolamine (DSPE), 16-O-monomethyl-phospho ethanolamine, 16-O-dimethyl-phosphoethanolamine, 18-1-trans-phosphoethanolamine, 1-stearoyl-2-oleoyl phosphatidyethanolamine (SOPE), and 1,2-dioleoyl-sn glycero-3-phophoethanolamine (transDOPE). In some embodiments, component (ii) excludes a polyalkylene oxide segment. In some embodiments, the non-cationic lipid in component (ii) is a phospholipid, or more particularly, a phosphatidyl serine lipid. In some embodiments, the cationic or ionizable lipid in component (iii) possesses a secondary, tertiary, or quaternary amino group, any of which may or may not be attached to a lipid moiety. In other embodiments, the cationic or ionizable lipid in component (iii) possesses a secondary, tertiary, or quaternary group along with a functional group which is negatively charged under physiological conditions. In some embodiments, the cationic or ionizable lipid in component (iii) excludes a polyalkylene oxide segment. In yet other embodiments, the lipid nanoparticle composition further comprises: (v) cholesterol or a derivative thereof.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1D. In vivo luciferase expression after the delivery of MD1-LNP encapsulating Fluc-mRNA (0.2 mg/kg). FIG. 1A shows Luciferase expression in C57bl/6 mice at 6 h, 24 h and 48 h post-injection. For LipoD treated group (left), mice were pre-treated intravenously with clodronate liposome at a dosage of 1 ml/100 g body weight at 18 h before LNP injection. FIG. 1B compares ROI in MD1-LNP treated mice (6 h post LNP injection) with/without macrophage depletion. (p<0.05, t-test). FIG. 1C shows luciferase expression in organs (harvested at 6 h post LNP injection) with/without macrophage depletion. FIG. 1D shows luciferase expression in C57bl/6 mice at 6 h, 24 h and 48 h post MD1/PS-LNP injection. PS containing lipid further enhanced mRNA expression in mice treated by clodronate liposome.

FIGS. 2A-2B. In vivo luciferase expression of MD1/DDAB-LNP (0.2 mg/kg of mRNA encoding Fluc protein). FIG. 2A shows luciferase expression in C57bl/6 mice at 6 h, 24 h, 48 h and 72 h post MD1/DDAB-LNP injection. Upper group of mice were pre-treated intravenously with clodronate liposome at a dosage of 1 ml/100 g body weight at 18 h before LNP injection. FIG. 2B compares ROI in MD1-LNP treated mice (6 h post LNP injection) with/without macrophage depletion. (p<0.0001, t-test).

FIGS. 3A-3B. In vivo luciferase expression of MD1/DDab/PS-LNP (0.2 mg/kg of mRNA encoding Fluc protein). FIG. 3A shows luciferase expression in C57bl/6 mice at 6 h, 24 h, 48 h and 72 h post MD1/DDab/PS-LNP injection. Upper group of mice were pre-treated with clodronate liposome at a dosage of 1 ml/100 g body weight at 18 h before LNP injection. FIG. 3B compares ROI in MD1-LNP treated mice (6 h post LNP injection) with/without macrophage depletion, p<0.0001, t-test.

FIGS. 4A-4B. FIG. 4A shows in vivo hEPO expression of MD1/PS-LNP (0.2 mg/kg of mRNA encoding EPO protein) in C57bl/6 mice with or without clodronate liposome at a dosage of 1 ml/100 g body weight at 18 h before LNP injection. FIG. 4B compares hEPO expression delivered by MD1-, MC3-and MD1/PS LNP, respectively. All mice were pre-treated with LipoD as described above.

FIG. 5. Table showing screening of small molecule drugs and monoclonal antibodies with macrophage/monocytes depletion capabilities for enhanced mRNA delivery.

FIGS. 6A-6B. Luciferase expression of pretreated mice injected with MC3-LNP loaded with Fluc mRNA (0.2 mg/kg). FIG. 6A shows from left to right: mice without treatment, treated with clodronate liposome, anti-CD115 mAB, anti-CCR2, anti-Ly6C, anti-Gr-1, and zoledronate. FIG. 6B shows from left to right: mice without depletion, treated with zoledronate liposome (10 μg, 50 μg and 100 μg). As a result, clodronate liposome, Anti-CD115, 10 μg of zoledronate, 50 μg and 100 μg of zoledronate liposome enhanced the mRNA delivery of MC3-LNPs in mice livers.

FIGS. 7A-7C. In vivo Cre induced gene recombination in Ai14 mice (0.2 mg/kg Cre-mRNA loaded in MC3-LNP). Organs were isolated at 48 h after injections of LNPs. FIG. 7A shows fluorescent images of organs from non-treated mice (upper figure) and pre-treated mice (lower figure), respectively. In the pre-treated group, mice were injected with anti-CD115 monoclonal antibodies following an injection scheme as shown. Fluorescent images of main organs (heart, lung, superficial cervical lymph node, liver, kidneys and spleen) were obtained by IVIS imager. FIG. 7B shows liver sections from PBS treated (negative control), LNP-mRNA (no depletion treatment), LipoD+LNP and anti-CD115+LNP stained with DAPI and imaged by confocal laser scanning microscopy (CLSM). As a result, depletion of resident macrophages of liver enhanced the Cre-mRNA delivery. FIG. 7C shows mean fluorescence intensities obtained from CLSM images of mice liver sections. Compared to the no depletion treatment group, significant increased mean fluorescence intensities were observed in LipoD and anti-CD115 treated groups.

FIGS. 8A-8B. In vivo CRISPR/Cas9 mediated gene editing in Ai14 mice with MD-1 LNPs encapsulating Cas9 mRNA and sgRNA (total RNA 1.25 mg/kg, mRNA/sgRNA=4/1, wt/wt). Livers were harvested 10 days post initial LNP injections. Macrophage depletion was performed by injecting LipoD solution 18 h prior to LNP injection. FIG. 8A shows fluorescent images (top) of livers from PBS treated, MD1 LNP treated and MD1+LipoD treated mice, respectively, and associated fluorescence intensity data (bottom). Fluorescent images were obtained by IVIS imager. FIG. 8B shows liver sections from PBS treated, MD1 LNP (no depletion treatment), and MD1+lipoD mice were stained with DAPI and imaged by confocal laser scanning microscopy (CLSM). As a result, depletion of resident macrophages of liver enhanced the LNP-mRNA gene editing efficiency.

### DETAILED DESCRIPTION

The present disclosure is foremost directed to a method of delivering a therapeutic substance (e.g., a nucleic acid) to a subject in which monocytes and/or macrophages have been depleted. In the method, a lipid nanoparticle (LNP) composition loaded with the therapeutic substance is administered to a subject in which monocytes and/or macrophages have been depleted. In some embodiments, the method may be practiced by first depleting monocytes and/or macrophages in the subject followed by administering the LNP loaded with the therapeutic substance (e.g., a nucleic acid) to the subject, thereby delivering the therapeutic substance into the subject. The subject is typically a mammal, more typically a human subject, but may also be another type of mammal, such as a pet or farm animal, such as a primate, dog, cat, cow, sheep, mouse, rat, rabbit, horse, or pig.

In some embodiments, the method of delivering the therapeutic substance results in a method of treating the subject. As a method of treatment, the LNP composition, which is loaded with therapeutic substance (e.g., nucleic acid), can be administered for the purpose of, for example, protein replacement therapy, cancer immunotherapy, cancer vaccine therapy, infectious disease vaccines, gene editing, autoimmune disease treatment, and/or cancer diagnosis. In particular embodiments, the LNP composition is administered for gene therapy comprising CRISPR-Cas gene editing, or for in vitro and in vivo production of extracellular vesicles, or for vaccination against coronavirus (e.g., SARS-COV-2). In more particular embodiments, the LNP is used for clustered regularly interspaced short palindromic repeats-Cas endonuclease (CRISPR-Cas) gene editing in vitro and in vivo, for example including but not limited to delivering one or more nucleic acids that encode for one or more CRISPR associated proteins such as Cas protein. In some embodiments, the LNP is administered along with a checkpoint inhibitor (e.g., anti-Programmed death-ligand 1 (anti-PD-L1) antibody or anti-cytotoxic T-lymphocyte-associated protein 4 (anti-CTLA4) to treat cancer.

In some embodiments, the method of treatment includes targeted delivery of a therapeutic agent to a secondary lymphoid organ (SLO) in a subject, wherein the subject is administered lipid nanoparticles comprising a phosphoserine-containing lipid and the therapeutic agent. The SLO may be, for example, spleen and/or lymph nodes. The phosphoserine-containing lipid may be, for example, 1,2-dioleoyl-sn-glycero-3-phospho-L-serine (DOPS), or a naturally-occurring PS-lipid, such as L-α-phosphatidylserine (brain). In some embodiments, the targeted delivery results in cancer immunotherapy, autoimmune disease immunotherapy, or gene editing.

The LNP composition is typically administered in the form of a pharmaceutical composition containing the LNP. In the pharmaceutical composition, the LNP may be dissolved or suspended in, or admixed with, a pharmaceutically acceptable carrier, which may be a liquid, semi-solid (e.g., gel or wax), or solid, as well known in the art. The phrase “pharmaceutically acceptable” refers herein to those compounds, materials, compositions, and/or dosage forms which are, within the scope of sound medical judgment, suitable for administration to a subject. Each carrier should be “acceptable” in the sense of being compatible with the other ingredients of the formulation and physiologically safe to the subject. Any of the carriers known in the art can be suitable herein depending on the mode of administration.

Some examples of pharmaceutically acceptable liquid carriers include alcohols (e.g., ethanol), glycols (e.g., propylene glycol and polyethylene glycols), polyols (e.g., glycerol), oils (e.g., mineral oil or a plant oil), paraffins, and aprotic polar solvents acceptable for introduction into a mammal (e.g., dimethyl sulfoxide or N-methyl-2-pyrrolidone) any of which may or may not include an aqueous component (e.g., at least, above, up to, or less than 10, 20, 30, 40, or 50 vol % water). Some examples of pharmaceutically acceptable gels include long-chain polyalkylene glycols and copolymers thereof (e.g., poloxamers), cellulosic and alkyl cellulosic substances (as described in, for example, U.S. Pat. No. 6,432,415), and carbomers. The pharmaceutically acceptable wax may be or contain, for example, carnauba wax, white wax, bees wax, glycerol monostearate, glycerol oleate, and/or paraffins.

In some embodiments, the pharmaceutical composition contains solely the LNP and one or more solvents or the carrier. In other embodiments, the pharmaceutical composition includes one or more additional components. The additional component may be, for example, a pH buffering agent, mono-or poly-saccharide (e.g., lactose, glucose, sucrose, trehalose, lactose, or dextran), preservative, electrolyte, surfactant, or antimicrobial. If desired, a sweetening, flavoring, or coloring agent may be included. Other suitable excipients can be found in standard pharmaceutical texts, e.g. in “Remington's Pharmaceutical Sciences”, The Science and Practice of Pharmacy, 19th Ed. Mack Publishing Company, Easton, Pa., 1995.

The LNP composition, typically in the form of a pharmaceutical composition in which the loaded LNP is admixed with or suspended in a liquid or solid pharmaceutically acceptable carrier, can be administered to the subject by any suitable route. The LNP may be administered intravenously, orally, intramuscularly, intradermally, subcutaneously, intranasally, or by inhalation. In particular embodiments, the LNP is administered by injection into the subject. In some embodiments, the LNP is delivered to cells of the subject. In some embodiments, the LNP is delivered by removing cells from the subject, administering the lipid nanoparticle to the removed cells, and then reintroducing the removed cells to the subject. In some embodiments, the LNP is injected directly in vivo and delivered into the host cells in vivo. In other embodiments, the LNP is transfected into the host cells ex vivo and the resulting cells are then infused in vivo.

As noted earlier above, by temporarily depleting monocytes and/or macrophages, more LNPs are advantageously permitted to accumulate in desirable tissues and or targeted cells, thereby resulting in enhanced nucleic acid (e.g., mRNA) delivery or maintained efficacy with lowered dose. In embodiments of the method, the subject is pre-treated by depleting monocytes and/or macrophages in the subject, i.e., before the subject is administered or otherwise treated with the loaded LNP composition described above. In some embodiments, the depletion of monocytes and/or macrophages is achieved by administering to the subject small molecule drugs having this ability. Some examples of small molecule drugs include clodronate, zoledronate, etidronate, tiludronate, pamidronate, neridronate, olpadronate, alendronate, ibandronate, and risedronate. In other embodiments, the depletion of monocytes and/or macrophages is achieved by administering to the subject liposome compositions loaded with small molecule drugs having this ability. Some examples of such liposome compositions include liposome clodronate (LipoD) and liposome zoledronate. In other embodiments, the depletion of monocytes and/or macrophages is achieved by administering to the subject antibodies configured to deplete monocytes and/or macrophages. Some examples of antibodies having this ability include anti-CD115, anti-CCR2, anti-Ly6C, anti-Gr-1. The depletion of monocytes and/or macrophages may be achieved within hours (e.g., 1, 2, 3, 4, 5, 6, 12, 18, or 24 hours, or range therein), within days (e.g., 1, 2, 3, 4, 5, 6, or 7 days, or range therein), or within weeks (e.g., 1, 2, 3, or 4 weeks), and the LNP may be administered to the subject after any one of the periods of time exemplified above for achieving depletion of monocytes and/or macrophages.

In some embodiments, the nucleic acid encodes a protein that is expressed in the subject. In some embodiments, the nucleic acid is a component of a gene editing machinery. In some embodiments, the nucleic acid is delivered into one or more of liver, brain, spleen, lymph nodes, kidneys, and lungs. The nucleic acid may be delivered into any biological tissue of the subject, including extracellular fluid (e.g., blood or blood plasma, i.e., systemically) and/or cells of the subject, wherein the cells may or may not be within organs or blood of the subject. The enhanced delivery of nucleic acid into the subject, as provided by the method described above, may result in an improved expression of protein encoded by the delivered nucleic acid. Alternatively or in addition, the enhanced delivery of nucleic acid into the subject may result in an improved gene editing efficiency by the delivered nucleic acid. Alternatively or in addition, the enhanced delivery of nucleic acid into the subject may enhance nucleic acid delivery into one or more of liver, brain, spleen, lymph nodes, kidneys, and lungs. The term “enhanced” or “enhancement,” as used herein, indicates the improvement in delivery of the nucleic acid achieved by the present method (i.e., wherein monocytes and/or macrophages have been depleted in the subject) compared to the delivery of the nucleic acid to a subject in which monocytes and/or macrophages have not been depleted. In embodiments, the enhancement may be quantified as an at least or greater than 10%, 20%, 30%, 40%, 50%, 60%, 70%, 80%. 90%, 100%, 120%, 150%, 180%, or 200% enhancement. In some embodiments, the nucleic acid encodes a protein, and the protein expression is enhanced by any of the values provided above. In other embodiments, the nucleic acid (e.g., mRNA) is a component of a gene editing machinery, and the gene editing is enhanced by any of the values provided above. In some embodiments, the nucleic acid is delivered into one or more of liver, brain, spleen, lymph nodes, kidneys, and lungs, and the delivery is enhanced by any of the values provided above.

The LNP may be any of the LNPs known in the art or as further described in greater detail below. Some LNPs of the art are described in, for example, X. Hou et al., Nature Reviews Materials, 6, 1078-1094, 2021, the contents of which are herein incorporated by reference. The term “lipid nanoparticle” refers to nanoparticles constructed, at least in part, of lipid molecules. As further described below, in some embodiments, the lipid nanoparticles include one or more zwitterionic polymer-containing lipids described herein, one or more non-cationic lipids, one or more cationic or ionizable lipids, and/or cholesterol. In different embodiments, the LNP has a size of precisely, about, at least, or up to, for example, 10, 20, 30, 40, 50, 60, 70, 80, 90, 100, 125, 150, 175, 200, 225, or 250 microns, or a size within a range bounded by any two of the foregoing values. The LNP is loaded with a therapeutic substance, particularly a nucleic acid substance, as further discussed below.

In some embodiments, the LNP contains at least one zwitterionic polymer-containing lipid. In the zwitterionic polymer-containing lipid, a lipid moiety is covalently attached (i.e., linked) to a zwitterionic polymer. The zwitterionic polymers are generally derived from zwitterionic monomers, as well as monomers that can be converted to zwitterionic monomers, i.e., precursors of zwitterionic monomers. Zwitterionic monomers are electronically neutral monomers that include equal numbers of positive and negative charges (e.g., one of each). In some embodiments, the zwitterionic polymer contains a plurality of repeating units, each repeating unit comprising one positive and one negative charged moiety. The zwitterionic polymer typically contains at least or greater than 2, 5, or 10, and up to or less than 100, 200, 300, 400, 500, or 1000 units.

As used herein, the term “zwitterionic polymer” refers to a polymer prepared by polymerizing a polymerizable zwitterionic monomer, which provides a zwitterionic polymer having 100 mole percent zwitterionic moieties, i.e., each repeating unit of the zwitterionic polymer is a zwitterionic moiety; or refers to a polymer prepared by copolymerizing a polymerizable zwitterionic monomer and a polymerizable comonomer, which provides a zwitterionic polymer having less than 100 mole percent zwitterionic moieties (e.g., when the polymerizable zwitterionic monomer and the polymerizable comonomer are present in equal proportions in the polymerization mixture, the product is a zwitterionic polymer having 50 mole percent zwitterionic moieties).

The term “zwitterionic polymer” also refers to a polymer having a substantially equal number of negative (anionic) charges and positive (cationic) charges that is prepared by copolymerizing a polymerizable negatively charged monomer and a polymerizable positively charged monomer, each present in substantially equal proportions in the polymerization mixture. The product of such a copolymerization is a zwitterionic polymer having 100 mole percent zwitterionic moieties, where each zwitterionic moiety is defined as a pair of repeating units: a repeating unit having a negative charge and a repeating unit having a positive charge. Such zwitterionic polymers are referred to as mixed charge copolymers. The term “zwitterionic polymer” also refers to a polymer prepared by copolymerizing a polymerizable negatively charged monomer, a polymerizable positively charged monomer, each present in substantially equal proportions in the polymerization mixture, and a polymerizable comonomer, which provides a zwitterionic polymer having less than 100 mole percent zwitterionic moieties. The mixed charge copolymer may contain, for example, 50% of a combination of polymerizable negatively charged monomer and polymerizable positively charged monomer and 50% of a polymerizable comonomer, which corresponds to a mixed charge copolymer product having 50 mole percent zwitterionic moieties.

The lipid moiety is constructed of a polyol portion (e.g., a diol, glycerol, phosphatidylglycerol, phosphatidylethanolamine, or phosphatidylserine) that has been esterified with one or two fatty acid molecules to result in a monoacyl or diacyl lipid, wherein the term “acyl” refers to a RC(═O) group in which R is a linear or branched hydrocarbon (fatty) chain containing at least eight and typically up to 30 carbon atoms, wherein the hydrocarbon chain may be saturated or contain one or more carbon-carbon double bonds. The lipid moiety may be, for example, a diacyldiol (e.g., diacylethyleneglycol), diacylglycerol (diacylglyceride), diacylphosphatidylglycerol, diacylphosphatidylethanolamine, or diacylphosphatidylserine moiety. The lipid may be any of the lipids described in any one of Examples 1-14 provided in this application. The lipid may also be any of the lipids described in WO2011/057227, which is herein incorporated by reference. The fatty acyl portion may be derived from any of the known fatty acids. Some examples of fatty acyl portions include oleoyl, palmitoyl, lauryl, myristoyl, stearoyl, linoleoyl, and arachidonyl. The zwitterionic polymer is attached to the lipid moiety, such as any of the lipid moieties described above, typically via a carbon on the polyol. Modes and methods of attaching the lipid to the zwitterionic polymer are provided in detail in, for example, U.S. Pat. No. 8,617,592, the contents of which are herein incorporated by reference. For example, a zwitterionic polymer (e.g., PCB) may be functionalized with a succinimide group by methods well known in the art, followed by reaction with an amino-functionalized lipid (e.g., DSPE) to attach the lipid to the zwitterionic polymer. The zwitterionic polymer may contain the zwitterionic groups in side chains or the backbone (or combination thereof) of the polymer.

In one embodiment, the zwitterionic polymer is a homopolymer prepared from zwitterionic monomers and has the formula:

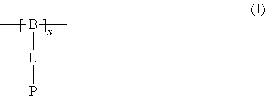

wherein B is a polymer backbone, L is an optional linker, and P is a zwitterionic moiety. The backbone (B) can be any polymeric backbones known in the art, including linear, branched, and cyclic backbone structures, e.g., a polyester, polyether, polyurethane, polyamide, or polyhydrocarbon (e.g., polyethylene or polypropylene) backbone. The linker (L) can be any of the linkers commonly included in pendant groups of polymers, e.g., linear or branched alkylene linkers or cyclic linkers, any of which may contain precisely or at least 1, 2, 3, 4, 5, or 6 carbon atoms and optionally containing one or more heteroatoms (typically selected from oxygen and nitrogen atoms). The subscript x is typically at least or greater than 2, 5, 10, 20, 50, or 100, and up to or less than 100, 200, 300, 400, 500, 1000, 2000, or 5000 units.

In particular embodiments, the backbone (B) may have any of the following structures:

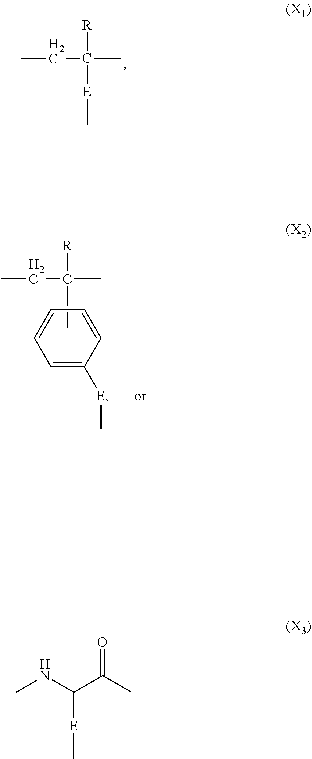

wherein R is selected from hydrogen atom and substituted or unsubstituted alkyl; and E is selected from substituted or unsubstituted alkylene, —(CH₂)ₚC(O)O—, and —(CH₂)ₚC(O)NR²—, wherein p is typically an integer from 0 to 12 and R² is selected from hydrogen and substituted or unsubstituted alkyl.

In particular embodiments, P is selected from any of the following structures:

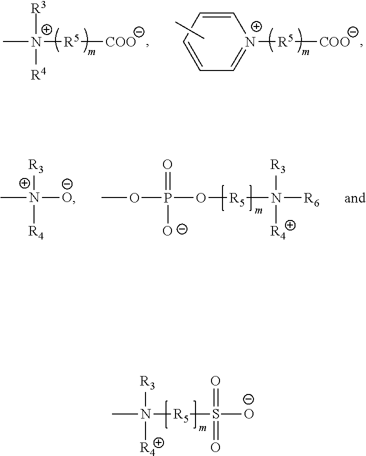

wherein R³, R⁴, and R⁶ are independently selected from the group consisting of hydrogen and substituted or unsubstituted alkyl group, R⁵ or R₅ is selected from the group consisting of substituted or unsubstituted alkylene, phenylene, and polyether groups, and m is an integer from 1 to 7; and x is an integer from 2 to 500.

In one set of embodiments, the zwitterionic polymer has the following structure:

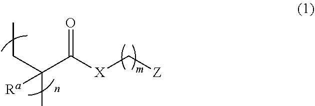

In Formula (1) above, the variable R^{a} is H or an alkyl group containing 1-3 carbon atoms. Some examples of alkyl groups containing 1-3 carbon atoms include methyl, ethyl, n-propyl, and isopropyl. In some embodiments, R^{a} is H or methyl. The variable X is O or NR^{b}, wherein R^{b} is H or an alkyl group containing 1-3 carbon atoms. In some embodiments, R^{b} is H or methyl. The variable Z is a zwitterionic moiety. The subscript n is typically an integer of at least 2, 5, or 10 (e.g., at least or greater than 10, 50, 100, 200, 300, 400, 500, 1000, 5000, 10,000, 50,000, or 100,000 units, or a number of units within a range bounded by any two of the foregoing values). The subscript m is an integer of at least 1, such as a value of precisely or at least 1, 2, 3, 4, 5, 6, 7, 8, 9, 10, 11, or 12, or a value within a range bounded by any two of the foregoing values (e.g., 1-12, 1-10, 1-6, 1-4, 1-3, 2-4, or 2-3).

In some embodiments, the group Z represents a single zwitterionic group containing a positive and negative charge in the same Z group. The group Z may be selected from, for example, carboxybetaine, sulfobetaine, phosphobetaine, and trialkylamine-N-oxide zwitterionic moieties. The resulting polymer according to Formula (1) is poly (carboxybetaine), poly (sulfobetaine), poly (phosphobetaine), and poly (trialkylamine-N-oxide), as well known in the art. In other embodiments, a certain number of the Z groups are positively charged and an equal number of Z groups are negatively charged to result in Z zwitterionic pairs (i.e., Z⁺Z⁻ zwitterionic pairs).

Notably, although Formulas (I), (1), and sub-formulas thereof may appear to depict zwitterionic homopolymers (100 mol % zwitterionic moieties), Formulas (I), (1), and sub-formulas thereof include the possibility that one or more non-zwitterionic or uncharged monomer units is situated (inserted) between zwitterionic monomeric units depicted in any of these formulas, thereby resulting in a copolymer. If zwitterionic monomeric units (such as any of those described above, i.e., where n=1) are labeled as A units, and non-zwitterionic or uncharged monomeric units are labeled as B units, the copolymer can have any of the known copolymer arrangements, including alternating (e.g., A-B-A-B), block (e.g., A-A-A-A-B-B-B-B), or random (e.g., A-B-B-A-B-A-A-B-A-B-B). The zwitterionic polymer may also include more than one type of non-zwitterionic or uncharged monomer unit, such as in the structures A-B-C-A-B-C (repeating), A-A-A-B-B-B-C-C-C (block), or A-C-B-B-C-A-B-C-A-C-A-B-C-A-B-C-B (random), wherein B and C represent non-zwitterionic or uncharged monomeric units.

In some embodiments of Formula (1), Z contains a positively charged group directly bound to a negatively charged group in the zwitterionic polymer. The resulting zwitterionic polymer may have the following structure:

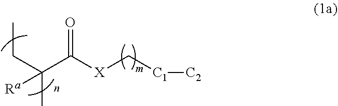

In Formula (1a), R^{a}, X, n, and m are as defined above under Formula (1). The variables C₁ and C₂ are independently selected as positively charged and negatively charged moieties to form a zwitterionic moiety C₁-C₂. Some examples of positively charged moieties include ammonium (—NR^{a}₂⁺—) and phosphonium (—PR^{a}₂⁺—) moieties. Some examples of negatively charged moieties include terminal oxide (—O⁻), carboxylate (—C(O)O—), phosphate (—OPO₃⁻), phosphonate (—PO₃⁻), sulfate (—OSO₃⁻), and sulfonate (—SO₃⁻). In some embodiments, C₁ is positively charged and C₂ is negatively charged. For example, C₁ may be an ammonium moiety and C₂ may be oxide, which together results in an ammonium N-oxide (—NR^{a}₂⁺—O⁻) zwitterionic group. As the ammonium moiety is also attached to a carbon atom of the polymer, the ammonium oxide zwitterionic group is also herein referred to as a trialkylamine-N-oxide group. In specific embodiments, m has a value of 1, 2, 3, or 4. In separate or further specific embodiments, R^{a} is H or methyl. In other separate or further specific embodiments, X is O or NR^{b}, wherein R^{b} is H or an alkyl group containing 1-3 carbon atoms, or R^{b} is H or methyl.

In specific embodiments of Formula (1a), C₁ is an ammonium moiety and C₂ is oxide, which together results in an ammonium N-oxide (—NR^{a}₂⁺—O⁻) zwitterionic group. The resulting polymer is a poly (trialkylammonium oxide), i.e., pTMAO, and may have the following structure:

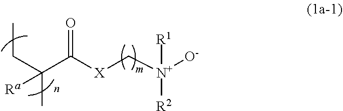

In Formula (1a-1), R^{a}, X, n, and m are as defined above under Formula (1). The variables R¹ and R² are independently selected from R^{a}. In some embodiments, R¹ and R² are both alkyl, or more particularly, both methyl. In specific embodiments, m has a value of 1, 2, 3, or 4. In separate or further specific embodiments, R^{a} is H or methyl. In separate or further specific embodiments, X is O or NR^{b}, wherein R^{b} is H or an alkyl group containing 1-3 carbon atoms, or R^{b} is H or methyl.

In other embodiments of Formula (1), Z contains a positively charged group

indirectly bound to a negatively charged group via a linker in the zwitterionic polymer. The resulting zwitterionic polymer may have the following structure:

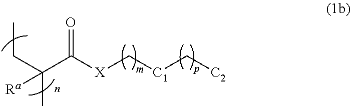

In Formula (1b), R^{a}, X, n, and m are as defined above under Formula (1). The subscript p is an integer of at least 1, such as a value of precisely or at least 1, 2, 3, 4, 5, 6, 7, 8, 9, 10, 11, or 12, or a value within a range bounded by any two of the foregoing values (e.g., 1-12, 1-10, 1-6, 1-4, 1-3, 2-4, or 2-3). The variables C₁ and C₂ are independently selected as positively charged and negatively charged moieties to form a zwitterionic spaced pair. Some examples of positively charged moieties include ammonium (—NR^{a}₂⁺—) and phosphonium (—PR^{a}₂⁺—) moieties. Some examples of negatively charged moieties include terminal oxide (—O⁻), carboxylate (—C(O)O—), phosphate (—OPO₃⁻), phosphonate (—PO₃⁻), sulfate (—OSO₃⁻), and sulfonate (—SO₃⁻). In some embodiments, C₁ is positively charged and C₂ is negatively charged. For example, C₁ may be an ammonium or phosphonium moiety and C₂ may be carboxylate, sulfate, sulfonate, phosphate, or phosphonate moiety. In other embodiments, C₁ is negatively charged and C₂ is positively charged. For example, C₁ may be a phosphate, phosphonate, sulfate, or sulfonate moiety and C₂ may be an ammonium or phosphonium moiety. In specific embodiments, m has a value of 1, 2, 3, or 4. In separate or further specific embodiments, p has a value of 1, 2, 3, or 4, or a value of 2, 3, or 4. In separate or further specific embodiments, R^{a} is H or methyl. In other separate or further specific embodiments, X is O or NR^{b}, wherein R^{b} is H or an alkyl group containing 1-3 carbon atoms, or R^{b} is H or methyl.

In specific embodiments of Formula (1b), C₁ is an ammonium moiety and C₂ is a negatively charged moiety, such as a carboxylate, sulfate, sulfonate, phosphate, or phosphonate moiety, which together results in a spaced zwitterionic group. The resulting zwitterionic polymer may have the following structure:

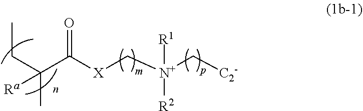

In Formula (1b-1), R^{a}, X, n, m, and p are as defined above under Formulas (1) and (1b). The variables R¹ and R² are independently selected from R^{a}. In some embodiments, R¹ and R² are both alkyl, or more particularly, both methyl. C₂ is a negatively charged moiety, such as a carboxylate, phosphate, phosphonate, sulfate, or sulfonate moiety. In specific embodiments, m has a value of 1, 2, 3, or 4. In separate or further specific embodiments, p has a value of 1, 2, 3, or 4, or a value of 2, 3, or 4. In separate or further specific embodiments, R^{a} is H or methyl. In separate or further specific embodiments, X is O or NR^{b}, wherein R^{b} is H or an alkyl group containing 1-3 carbon atoms, or R^{b} is H or methyl. In embodiments where C₂ is a carboxylate moiety, the polymer of Formula (1b-1) can generally be referred to as a poly (carboxybetaine). In embodiments where C₂ is a sulfonate moiety, the polymer of Formula (1b-1) can generally be referred to as a poly(sulfobetaine). In embodiments where C₂ is a phosphate moiety, the polymer of Formula (1b-1) can generally be referred to as a poly(phosphobetaine).

In specific embodiments of Formula (1b-1), C₂ is a sulfonate or carboxylate moiety. The resulting zwitterionic polymer may have any of the following structures:

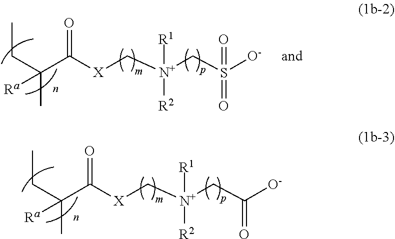

In Formulas (1b-2) and (1b-3), R^{a}, X, n, m, and p are as defined above under Formulas (1) and (1b). The variables R¹ and R² are independently selected from R^{a}. In some embodiments, R¹ and R² are both alkyl, or more particularly, both methyl. In separate or further specific embodiments, m has a value of 1, 2, 3, or 4. In separate or further specific embodiments, p has a value of 1, 2, 3, or 4, or a value of 2, 3, or 4. In separate or further specific embodiments, R^{a} is H or methyl. In separate or further specific embodiments, X is O or NR^{b}, wherein R^{b} is H or an alkyl group containing 1-3 carbon atoms, or R^{b} is H or methyl.

In other specific embodiments of Formula (1b), C₁ is a phosphonium moiety and C₂ is a negatively charged moiety, such as a carboxylate, sulfate, sulfonate, phosphate, or phosphonate moiety, which together results in a spaced zwitterionic group. The resulting zwitterionic polymer may have the following structure:

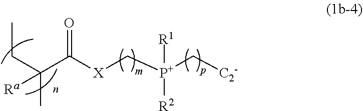

In Formula (1b-4), R^{a}, X, n, m, and p are as defined above under Formulas (1) and (1b). The variables R¹ and R² are independently selected from R^{a}. In some embodiments, R¹ and R² are both alkyl, or more particularly, both methyl. C₂ is a negatively charged moiety, such as a carboxylate, phosphate, phosphonate, sulfate, or sulfonate moiety. In specific embodiments, m has a value of 1, 2, 3, or 4. In separate or further specific embodiments, p has a value of 1, 2, 3, or 4, or a value of 2, 3, or 4. In separate or further specific embodiments, R^{a} is H or methyl. In separate or further specific embodiments, X is O or NR^{b}, wherein R^{b} is H or an alkyl group containing 1-3 carbon atoms, or R^{b} is H or methyl.

In specific embodiments of Formula (1b-4), C₂ is a sulfonate or carboxylate moiety. The resulting zwitterionic polymer may have any of the following structures:

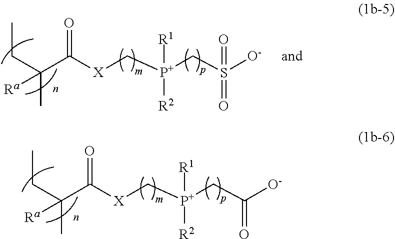

In Formulas (1b-5) and (1b-6), R^{a}, X, n, m, and p are as defined above under Formulas (1) and (1b). The variables R¹ and R² are independently selected from R^{a}. In some embodiments, R¹ and R² are both alkyl, or more particularly, both methyl. In separate or further specific embodiments, m has a value of 1, 2, 3, or 4. In separate or further specific embodiments, p has a value of 1, 2, 3, or 4, or a value of 2, 3, or 4. In separate or further specific embodiments, R^{a} is H or methyl. In separate or further specific embodiments, X is O or NR^{b}, wherein R^{b} is H or an alkyl group containing 1-3 carbon atoms, or R^{b} is H or methyl.

In other specific embodiments of Formula (1b), C₁ is a phosphate moiety and C₂ is a positively charged moiety, such as an ammonium or phosphonium moiety, which together results in a spaced zwitterionic group. The resulting zwitterionic polymer may have the following structure:

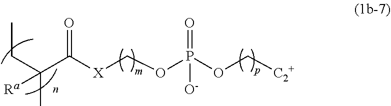

In Formula (1b-7), R^{a}, X, n, and m are as defined above under Formula (1). The subscript p is an integer of at least 1, such as a value of precisely or at least 1, 2, 3, 4, 5, 6, 7, 8, 9, 10, 11, or 12, or a value within a range bounded by any two of the foregoing values (e.g., 1-12, 1-10, 1-6, 1-4, 1-3, 2-4, or 2-3). The variable C₂⁺ is a positively charged moiety that forms a zwitterionic spaced pair with the phosphate moiety in Formula (1b-7). Some examples of positively charged moieties include ammonium (—NR^{a}₂⁺—) and phosphonium (—PR^{a}₂⁺—) moieties. In specific embodiments, m has a value of 1, 2, 3, or 4. In separate or further specific embodiments, p has a value of 1, 2, 3, or 4, or a value of 2, 3, or 4. In separate or further specific embodiments, R^{a} is H or methyl. In other separate or further specific embodiments, X is O or NR^{b}, wherein R^{b} is H or an alkyl group containing 1-3 carbon atoms, or R^{b} is H or methyl.

In specific embodiments of Formula (1b-7), C₂⁺ is an ammonium moiety. The resulting zwitterionic polymer may have the following structure:

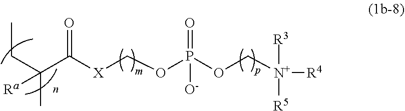

In Formula (1b-8), R^{a}, X, n, m, and p are as defined above under Formulas (1) and (1b). The variables R³, R⁴, and R⁵ are independently selected from R^{a}. In some embodiments, at least one or two of R³, R⁴, and R⁵ are alkyl (or more specifically, methyl and/or ethyl) or all of R³, R⁴, and R⁵ are alkyl (or more specifically, methyl and/or ethyl). In separate or further specific embodiments, m has a value of 1, 2, 3, or 4. In separate or further specific embodiments, p has a value of 1, 2, 3, or 4, or a value of 2, 3, or 4. In separate or further specific embodiments, R^{a} is H or methyl. In separate or further specific embodiments, X is O or NRb, wherein R^{b} is H or an alkyl group containing 1-3 carbon atoms, or R^{b} is H or methyl.

Some specific examples of zwitterionic polymers within the above formulas include:

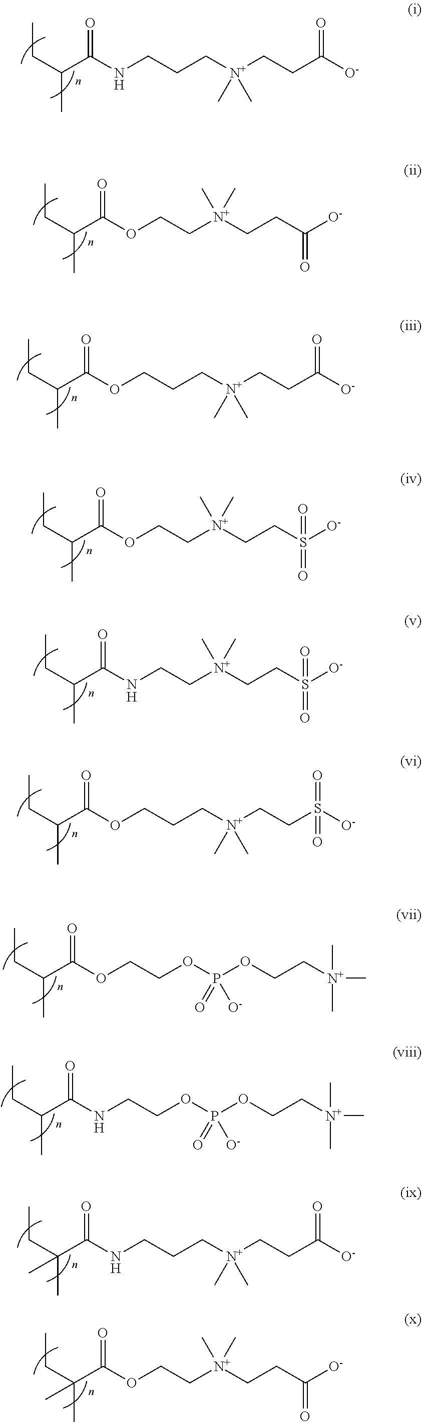

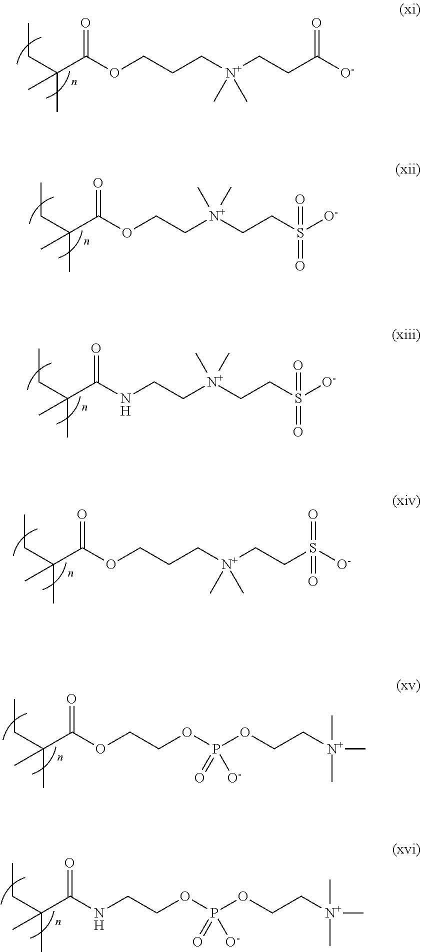

The zwitterionic polymer may alternatively be within the scope of Formula (I) but not within the scope of Formula (1) or sub-formulas thereof. Some examples of zwitterionic polymers not within the scope of Formula (1) or sub-formulas thereof but which may be included in the lipid nanoparticle composition include the following, wherein q can have any of the values given above for m (e.g., at least 1):

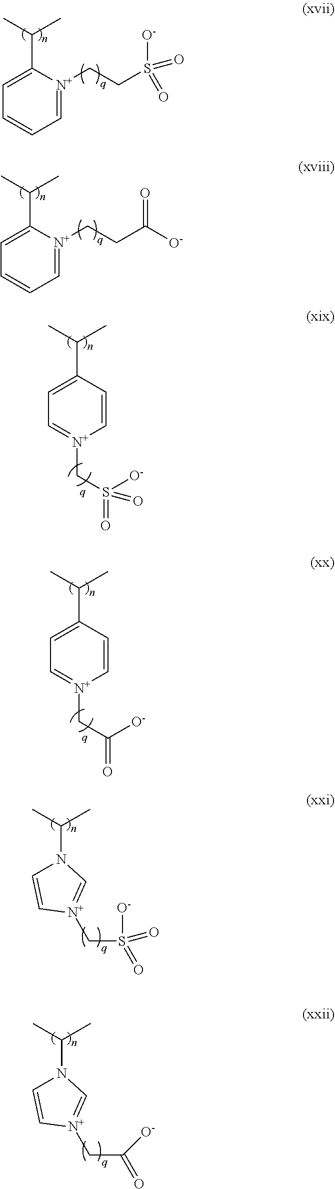

In some embodiments, the zwitterionic polymer is a betaine polymer. In other embodiments, the zwitterionic polymer is a poly(phosphatidylcholine) polymer, poly(trimethylamine N-oxide) polymer, poly(zwitterionic phosphatidylserine) polymer, or glutamic acid-lysine (EK)-containing polypeptide. In some embodiments, zwitterionic phosphatidyl serine comprises one neighboring positive charged moiety to balance the negative charge of the phosphoserine. In some embodiments, zwitterionic phosphatidyl serine comprises a compound as described in “De novo design of functional zwitterionic biomimetic material for immunomodulation” Science Advances, 29 May 2020, Vol. 6, Issue 22, (DOI: 10.1126/sciadv.aba0754) which is hereby incorporated by reference in its entirety.

Some examples of betaine polymers include poly(carboxybetaine), poly(sulfobetaine), and poly(phosphobetaine) polymers. Suitable poly(carboxybetaine) s can be prepared from one or more monomers selected from, for example, carboxybetaine acrylates, carboxybetaine acrylamides, carboxybetaine vinyl compounds, carboxybetaine epoxides, and mixtures thereof. In one embodiment, the monomer is carboxybetaine methacrylate. Representative monomers for making carboxybetaine polymers useful in the invention include carboxybetaine methacrylates, such as 2-carboxy-N,N-dimethyl-N-(2′-methacryloyloxyethyl) ethanaminium inner salt; carboxybetaine acrylates; carboxybetaine acrylamides; carboxybetaine vinyl compounds; carboxybetaine epoxides; and other carboxybetaine compounds with hydroxyl, isocyanates, amino, or carboxylic acid groups. In a particular embodiment, the polymer is a poly (carboxybetaine methacrylate) (poly(CBMA)).

In another embodiment, the zwitterionic polymer is a homopolymer that has a positive charge in the polymer backbone and a pendant carboxylic acid group and has the formula:

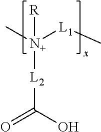

wherein R is selected from the group consisting of hydrogen and substituted or unsubstituted alkyl; L₁ and L₂ are independently a straight or branched alkylene group optionally including one or more oxygen atoms; and x is an integer from 2 to 500.

In another embodiment, the zwitterionic polymer is a mixed charge copolymer and has the general formula:

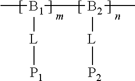

wherein B₁ and B₂ are independently selected from X₁, X₂, and X₃ as described earlier above; R is selected from hydrogen and substituted or unsubstituted alkyl; E is selected from substituted or unsubstituted alkylene, —(CH₂)ₚC(O)O—, and —(CH₂)ₚC(O)NR²—, wherein p is an integer from 0 to 12; R² is selected from hydrogen and substituted or unsubstituted alkyl; L is a straight or branched alkylene group optionally including one or more oxygen atoms; P₁ is a positively charged group; P₂ is a negatively charged group, such as a carboxylic acid group; m is an integer from 1 to 500; and n is an integer from 1 to 500. In some embodiments, P₁ is nitrogen in an aromatic ring or NR⁵R⁶, wherein R⁵ and R⁶ are independently substituted or unsubstituted alkyl group.

The positively charged unit (P₁ containing unit) of the zwitterionic polymer can be derived from a monomer having a positively charged pendant group. Representative monomers that can be used to derive the positively charged unit in the polymers of the present invention include 2-(dimethylamino) ethyl methacrylate, 2-(diethylamino)ethyl methacrylate, [2-(methacryloyloxy)ethyl]trimethylammonium chloride, and N-acetylglucosamine.

In one embodiment, the negatively charged unit of the zwitterionic polymer is derived from 2-carboxyethyl acrylate (CA), and the positively charged unit is derived from 2-(dimethylamino)ethyl methacrylate (DM). In another embodiment, the negatively charged unit is derived from 2-carboxyethyl acrylate (CA), and the positively charged unit is derived from 2-(diethylamino)ethyl methacrylate (DE). In another embodiment, the negatively charged unit is derived from 2-carboxyethyl acrylate (CA), and the positively charged unit is derived from [2-(methacryloyloxy) ethyl] trimethylammonium chloride (TM). In another embodiment, the negatively charged unit is derived from 2-carboxyethyl acrylate (CA), and the positively charged unit is derived from 2-aminoethyl methacrylate hydrochloride (NH₂).

In some embodiments, the zwitterionic polymer excludes a polyalkylene oxide (polyalkylene glycol) segment, or the zwitterionic polymer more specifically excludes a polyethylene oxide or polypropylene oxide segment. In some embodiments, all of component (i) excludes a polyalkylene oxide segment, or component (i) more specifically excludes a polyethylene oxide or polypropylene oxide segment. In some embodiments, the lipid nanoparticle as a whole excludes a polyalkylene oxide segment or molecule.

The zwitterionic polymer can be prepared by any suitable polymerization method, such as atom transfer radical polymerization (ATRP), reversible addition fragmentation chain transfer (RAFT) polymerization, and free radical polymerization. Any suitable radical initiators for polymerizing such monomers including those well known in the art, may be used. In some embodiments, to prepare the zwitterionic polymer-containing lipid or other polymer-containing lipid, a zwitterionic or other monomer or precursor thereof is attached to a lipid, and the monomer is polymerized while attached to the lipid. Alternatively, an already produced polymer may be attached to a lipid by means well known in the art.

The term “lipid” refers to organic compounds that include, but are not limited to, esters of fatty acids and are characterized by being insoluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids. In one embodiment, lipid includes diacylglyceride.

In one embodiment, the zwitterionic polymer is linked to “compound lipids”. Some examples of such lipids include dioleoylphosphatidylglycerol (DOPG), dipalmitoylphosphatidylglycerol (DPPG), dioleoylphosphatidylethanolamine (DOPE), palmitoyloleoylphosphatidylethanolamine (POPE), dipalmitoylphosphatidylethanolamine (DPPE), dimyristoylphosphoethanolamine (DMPE), distearoylphosphatidylethanolamine (DSPE), 16-O-monomethyl-phosphoethanolamine, 16-O-dimethyl-phosphoethanolamine, 18-1-trans-phosphoethanolamine, 1-stearoyl-2-oleoyl-phosphatidyethanolamine (SOPE), and 1,2-dioleoyl-sn-glycero-3-phophoethanolamine (transDOPE). In another embodiment, the zwitterionic polymer is linked to “simple lipids”. In another embodiment, the zwitterionic polymer is linked with “derived lipids”. In some embodiments, the zwitterionic polymer comprises zwitterionic compounds as disclosed in WO2011057225A2, which is incorporated herein by reference.

In some embodiments, at least one non-cationic lipid selected from charged and uncharged lipids not attached to a polymer is included as a second component of the LNP. The term “non-cationic lipid,” as used herein, refers to a lipid that is not positively charged and not capable of being ionized to a positively charged state. However, the non-cationic lipid may or may not be neutral charged by containing a zwitterion (positive and negative charge within the polymer), such as any of the zwitterionic groups and moieties described earlier above.

In some embodiments, the non-cationic lipid contains a zwitterionic moiety. The zwitterionic moiety can be any such moieties described in detail earlier above. The zwitterionic moiety may be, for example, a phosphobetaine, phosphatidylcholine, carboxybetaine, sulfobetaine, trimethylamine N-oxide, glutamic acid-lysine (EK)-containing, or zwitterionic phosphatidyl serine (phosphoserine) moiety, or a combination thereof. In some embodiments, the zwitterionic lipid is a phospholipid, such as a phosphatidylcholine or phosphatidylserine lipid.

In other embodiments, the non-cationic lipid is not zwitterionic but negatively charged by containing a negatively charged group (e.g., phosphoserine). A metal (e.g., alkali) or ammonium counteranion may be ionically and fluxionally associated with the negatively charged group.

In another set of embodiments, the non-cationic lipid is uncharged by not containing any charged groups. The uncharged non-cationic lipid may be, for example, a phosphatidylglycerol lipid, phosphatidylethanolamine lipid, or sphingolipid. The non-cationic lipid may, in some embodiments, be a simple lipid, such as a fat, oil, and/or wax. The non-cationic lipid may, in some embodiments, be a compound lipid, such as a phospholipid or glycolipid. The non-cationic lipid may, in some embodiments, be a derived lipid, such as a steroid, a phospholipid, a sphingolipid, and/or a sterol. In some embodiments, the non-cationic lipid is selected from a diacylphosphatidylethanolamine, a ceramide, a sphingomyelin, a dihydrosphingomyelin, a cephalin, or a cerebroside. In particular embodiments, the non-cationic lipid is selected from one or more of a phosphatidylethanolamine (PE), a phosphatidylglycerol (PG), a phosphatidic acid (PA), or a phosphatidylinositol (PI). In other particular embodiments, the non-cationic lipid is a dioleoylphosphatidylglycerol (DOPG), dipalmitoylphosphatidylglycerol (DPPG), dioleoylphosphatidylethanolamine (DOPE), palmitoyloleoyl-phosphatidylethanolamine (POPE), dipalmitoylphosphatidylethanolamine (DPPE), dimyristoylphosphoethanolamine (DMPE), distearoylphosphatidylethanolamine (DSPE), 16-O-monomethyl-phospho ethanolamine, 16-O-dimethyl-phosphoethanolamine, 18-1-trans-phosphoethanolamine, 1-stearoyl-2-oleoyl phosphatidyethanolamine (SOPE), and 1,2-dioleoyl-sn glycero-3-phophoethanolamine (transDOPE). Since the non-cationic lipid is not attached to a polymer, the non-cationic lipid excludes a polyalkylene oxide (polyalkylene glycol) segment.

In particular embodiments, non-cationic lipids containing an ionic moiety are phospholipids. In one embodiment, the non-cationic lipid containing an ionic moiety is a lipid conjugated with one or more carboxybetaine groups. In one embodiment, the non-cationic lipid containing an ionic moiety is a lipid conjugated with one or more sulfobetaine groups. In one embodiment, the non-cationic lipid containing an ionic moiety is a lipid conjugated with one or more trimethylamine N-oxide groups.

In some embodiments, at least one cationic or ionizable lipid is included as a third component of the LNP. The cationic or ionizable lipid may or may not contain a lipid attached to a polymer that has a cationic group. In some embodiments, the cationic or ionizable lipid is not attached to a polymer. The term “cationic lipid,” as used herein, refers to a positively charged lipid (typically, by possessing an ammonium group). In the cationic lipid, the positively charged group is not associated with a negative charge within the cationic lipid. Thus, the cationic lipid is not a zwitterionic lipid. The term “ionizable lipid,” as used herein, refers to lipids that contain one or more groups capable of being ionized to result in a positive charge in the polymer. The ionizable lipid generally possesses a secondary, tertiary, or quaternary amino group, or particularly an alkylated amine, or more particularly, a monoalkylamine or dialkylamine group, any of which can be protonated or alkylated to result in an alkylated ammonium group. The cationic lipid may contain a trialkylamine group, which is necessarily positively charged when bound to the lipid. In particular embodiments, the cationic or ionizable lipid possesses a dimethylamino or trimethylamino (or dimethylammonium or trimethylammonium) group. In particular embodiments, the cationic or ionizable lipids are selected from 1,2-dioleoyl-3-dimethylammonium-propane (DODAP), 1,2-dilinoleyloxy-N,N-dimethyl-3-aminopropane (DLinDMA), 2,2-dilinoleyl-4-(2-dimethylaminoethyl)-[1,3]-dioxolane (DLinKC2DMA), and [(6Z,9Z,28Z,31Z)-heptatriaconta-6,9,28,31-tetraen-19-yl] 4-(dimethylamino) butanoate (DLinMC3DMA).

In some embodiments, the ionizable lipid contains a lipid moiety attached to a secondary, tertiary or quaternary amine group along with a functional group, which is negatively charged under physiological conditions. This lipid moiety can have the following formula:

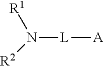

wherein:

R¹ and R² are independently selected from H and alkyl groups and wherein the alkyl group may be saturated, unsaturated, branched, and/or unbranched, and may optionally include one or more heteroatoms selected from N, O, F, Si, P, S, Cl, Br, and F;

L comprises a covalent linker group between N and A, wherein the covalent linker group is a linear or branched alkyl group containing 1-20 carbon atoms and may optionally include one or more heteroatoms selected from N, O, F, Si, P, S, Cl, Br, and F; The structure of L is exemplified by, but not limited to: —CH₂—, —CH₂CH(OH)—, —CH₂CHClCH₂—, —CH₂OCH₂—, —CH₂SCH₂—, —CH₂SSCH₂—, —CH₂COOCH₂—.

A is a functional group that is negatively charged or capable of being deprotonated to form a negatively charged group at a pH of 5-8. Structures are exemplified by, but not limited to the following:
(i) A is a carboxylic acid group (A=—COOH) or
(ii) A is a phosphate, i.e., where A=

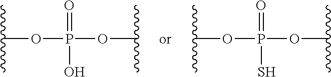 [SMILES: *OP(=O)(O)O* ; *OP(=O)(S)O*]

(iii) A is a sulfonic acid group, i.e., where A=

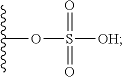 [SMILES: *OS(=O)(=O)O]

and
(iv) A is a sulfonamide group, i.e., where A=

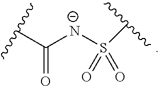 [SMILES: *C(=O)[N-]S(*)(=O)=O]

Exemplary structures of these cationic or ionizable lipids include:

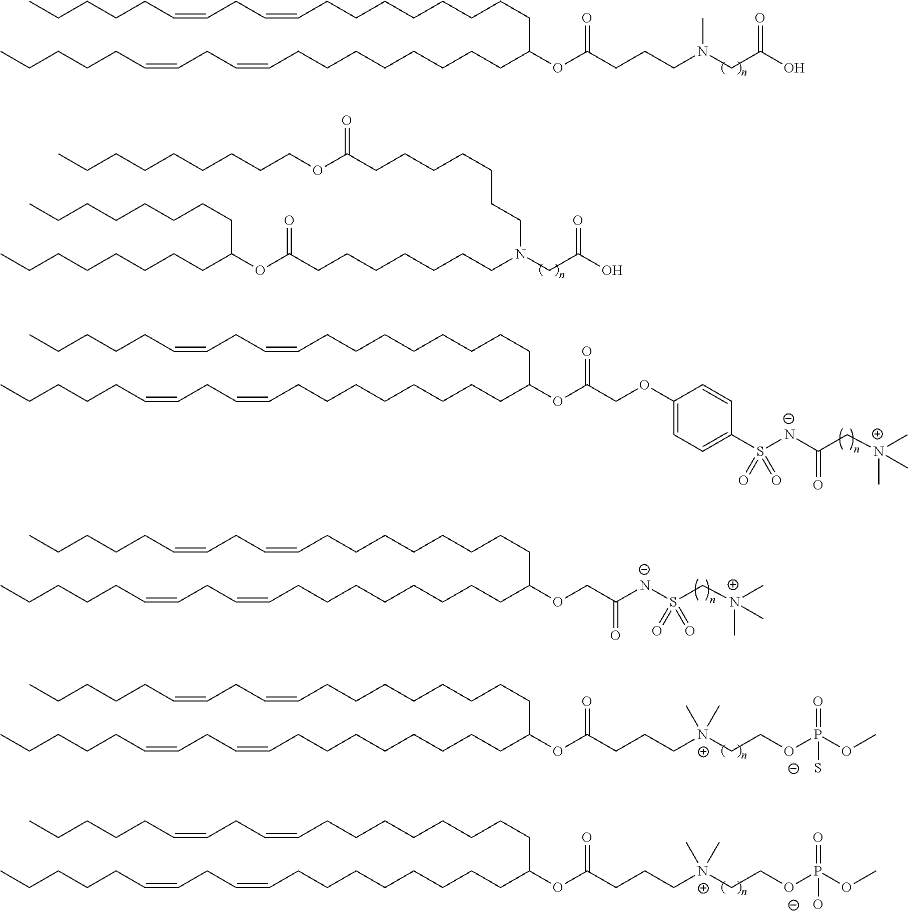

wherein n is typically a value in the range of 1-10.

In some embodiments, the cationic or ionizable lipid excludes a polyalkylene oxide (polyalkylene glycol) segment, or the cationic or ionizable lipid more specifically excludes a polyethylene oxide or polypropylene oxide segment. In some embodiments, the cationic or ionizable lipid excludes a polyalkylene oxide segment, or the cationic or ionizable lipid more specifically excludes a polyethylene oxide or polypropylene oxide segment. In some embodiments, the lipid nanoparticle excludes a polyalkylene oxide segment.

In some embodiments, the LNP further includes cholesterol or a derivative thereof, which is considered herein to be an optional further component of the LNP. In certain embodiments, the cholesterol derivative is a phytosterol, e.g., β-sitosterol, campesterol, stigmasterol, fucosterol, or stigmastanol. In certain embodiments, the cholesterol derivative is dihydrocholesterol, ent-cholesterol, epi-cholesterol, desmosterol, cholestanol, cholestanone, cholestenone, cholesteryl-2′-hydroxyethyl ether, cholesteryl-4′-hydroxybutyl ether, 3β[N-(N′N′-dimethylaminoethyl) carbamoyl cholesterol (DC-Chol), 24(S)-hydroxycholesterol, 25-hydroxycholesterol, 25(R)-27-hydroxycholesterol, 22-oxacholesterol, 23-oxacholesterol, 24-oxacholesterol, cycloartenol, 22-ketosterol, 20-hydroxysterol, 7-hydroxycholesterol, 19-hydroxycholesterol, 22-hydroxycholesterol, 25-hydroxycholesterol, 7-dehydrocholesterol, 5α-cholest-7-en-3β-ol, 3,6,9-trioxaoctan-1-ol-cholesteryl-3e-ol, dehydroergosterol, dehydroepiandrosterone, lanosterol, dihydrolanosterol, lanostenol, lumisterol, sitocalciferol, calcipotriol, coprostanol, cholecalciferol, lupeol, ergocalciferol, 22-dihydroegocalciferol, ergosterol, brassicasterol, tomatidine, tomatine, ursolic acid, cholic acid, chenodeoxycholic acid, zymosterol, diosgenin, fucosterol, fecosterol, or fecosterol, or a salt or ester thereof, e.g., sodium cholate.

The LNP further includes a therapeutic substance incorporated into or encapsulated by the self-assembled shell constructed of one or more of the first, second, third, and/or fourth lipid components described above. The therapeutic substance can be any substance having therapeutic value for a living organism, particularly a mammal, such as a human or animal subject. The therapeutic substance may be, for example, a negatively charged nucleic molecule. The nucleic molecule may be, for example, a nucleotide, nucleoside, nucleobase, or a nucleic acid (e.g., DNA or RNA). In particular embodiments, the therapeutic substance contains one or more such nucleic molecules. In some embodiments, the therapeutic substance contains RNA, or more particularly, mRNA, or more particularly viral mRNA. In other particular embodiments, the therapeutic substance is a spike protein of a virus, such as a coronavirus, SARS-COV2 (COVID-19), or HIV virus.

The lipid nanoparticles and compositions of the present invention may be used for a variety of purposes, including the delivery of nucleic acid molecules, ribonucleoprotein (RNP) and numerous other therapeutic substances. Examples of nucleic acids include messenger RNA (mRNA), ribosomal RNA (rRNA), transfer RNA (tRNA), antisense oligonucleotide (ASO), short interfering RNAs (siRNA), microRNA (miRNA), miRNA inhibitors (antagomirs/antimirs), messenger-RNA-interfering complementary RNA (micRNA), multivalent RNA, circular RNA (circRNA), crispr RNA (crRNA), long noncoding RNA (IncRNA), plasmid DNA, oligo DNA, and complementary DNA (cDNA). In particular embodiments, the therapeutic molecule comprises one or more of DNA, RNA, ssDNA, dsDNA, ssRNA, dsRNA, and hybrids thereof. In other embodiments, the therapeutic molecule comprises one or more of plasmid DNA or linearized DNA. In other embodiments, the therapeutic molecule comprises one or more of messenger RNA (mRNA), small interfering RNA (siRNA), microRNA (miRNA), circular RNA (circRNA), and/or long-noncoding RNA (IncRNA). In other embodiments, the therapeutic molecule comprises antisense oligonucleotide (ASO). In other embodiments, the therapeutic molecule comprises Cas nuclease mRNA and/or guide RNA nucleic acid. The guide RNA nucleic acid may be, for example, a single-guide RNA (sgRNA). In any of the foregoing embodiments, the therapeutic molecule comprises a vaccine against SARS-Cov-2, particularly wherein the vaccine is an mRNA vaccine or wherein the mRNA vaccine corresponds to a spike protein or portion thereof.

In any of the foregoing embodiments, the nucleotide may encode fusion biological moieties comprising protective domains and functional domains. In some embodiments, the functional domains are fused to the protective domains directly or via a linker consisting of amino acids. In further particular embodiments, the protective domain may comprise: a plurality of negatively charged amino acids (e.g., aspartic acid, glutamic acid, and derivatives thereof); a plurality of positively charged amino acids (e.g., lysine, histidine, arginine, and derivatives thereof); and a plurality of additional amino acids independently selected from the group consisting of proline, serine, threonine, asparagine, glutamine, glycine, and derivatives thereof, wherein the ratio of the number of positively charged amino acids to the number of positively charged amino acids is from about 1:0.5 to about 1:2. In some embodiments, the protective domain can be selected from other amino acid polymers (e.g., extended recombinant polypeptide (XTEN), proline-alanine-serine and elastin-like polypeptides). In any of the foregoing embodiments, the protective domain can be selected from natural half-life extension domains (e.g., Fc fragment).

The LNP may also include lipid components with combined functionality. In embodiments, any of the lipid components, including but not limited to the zwitterionic polymer-containing lipid, the non-cationic lipid, the cationic lipid, and the cholesterol and/or cholesterol derivative, can include one or more functionalities of a different lipid component. In embodiments, the zwitterionic polymer-containing lipid includes the functionality of a zwitterionic polymer-containing lipid and a cationic or ionizable lipid. In embodiments, any of the zwitterionic polymer-containing lipid, the non-cationic or ionizable lipid, the cationic lipid, and the cholesterol and/or cholesterol derivative can also include one or more functionalities of one or more of the zwitterionic polymer-containing lipid, the non-cationic lipid, the cationic lipid, and the cholesterol and/or cholesterol derivative. In embodiments, a lipid component can include the functionality of another lipid component, thereby eliminating the need for an additional lipid component with that functionality. In embodiments, the zwitterionic polymer-containing lipid can include the functionality of the non-cationic lipid, thereby eliminating or reducing the need for the non-cationic lipid. In embodiments, the zwitterionic polymer-containing lipid can include the functionality of the cationic lipid, thereby eliminating or reducing the need for the cationic lipid. In embodiments, the zwitterionic polymer-containing lipid can include the functionality of the cholesterol and/or cholesterol derivative, thereby eliminating or reducing the need for the cholesterol and/or cholesterol derivative.

In embodiments, any lipid component (zwitterionic polymer modified lipids, cationic lipids, non-cationic lipids, and cholesterol or its derivative) may be chemically combined with any other lipid components. In embodiments, the zwitterionic polymer modified lipid and cationic lipid are chemically combined into one lipid. In embodiments, the zwitterionic polymer modified lipid and non-cationic lipid are chemically combined into one lipid. In embodiments, the cationic lipid and non-cationic lipid are chemically combined into one lipid. In embodiments, the zwitterionic polymer modified lipid, cationic lipid and non-cationic lipid are chemically combined into one lipid. In embodiments, the zwitterionic polymer modified lipid and cholesterol or a derivative are chemically combined into one lipid. In embodiments, the zwitterionic polymer modified lipid, cationic lipid and cholesterol or a derivative are chemically combined into one lipid. In embodiments, the zwitterionic polymer modified lipid, non-cationic lipid and cholesterol or a derivative are chemically combined into one lipid. In embodiments, the zwitterionic polymer modified lipid, non-cationic lipid, non-cationic lipids and cholesterol or a derivative are chemically combined into one lipid. In other embodiments, each lipid component is separate, distinct, and not combined with another lipid component.

Any of the components of the lipid nanoparticle described above may be chemically modified to contain a targeting ligand (e.g., Fab or Fc). In particular embodiments, the zwitterionic polymer-containing lipid possesses a targeting ligand to deliver LNPs loaded with a therapeutic or diagnostic agent or both of them to a targeted area within the special organ in the body, such as a peptide (e.g., RGD), a lipid (e.g., phosphoserine-containing lipid), a protein (e.g., apolipoprotein E), an aptamer (e.g., anti-VEGF aptamer), a sugar (e.g., Sialic acid) and an antibody (e.g., anti-PD1) or antibody fragment (e.g., Fab or Fc).

Examples have been set forth below for the purpose of illustration and to describe the best mode of the invention at the present time. However, the scope of this invention is not to be in any way limited by the examples set forth herein.

### EXAMPLES

### Example 1

### Preparing LNP-mRNA Formulations by Microfluidic Mixing

Encapsulation of messenger RNA (mRNA) into lipid nanoparticle (LNP) was prepared by mixing individual lipid nanoparticle components and a corresponding mRNA in a microfluidic mixing device. Briefly, all of the lipid nanoparticle components were dissolved in ethanol, and separately, mRNA encoding firefly luciferase (Fluc), recombinant human erythropoietin (hEPO) or Cre (Cre recombinase) were dissolved in 50 mM citrate buffer (pH=3). Then, the aqueous phase and ethanol phase were mixed at a flow rate ratio of 3:1 in the microfluidic device. To remove residual organic solvents, the resulting LNP was washed with PBS in a 100-kDa centrifugal filter.

LNP formulations are listed as follows (showed in molar ratios):

TABLE 1

| Formulation | Molar ratio of each component |
|---|---|
| MC3-LNP | MC3/DSPC/Cholesterol/PEG-DMG2k = |
| | 50/10/38.5/1.5 |
| MD1-LNP | MD1/DOPE/Cholesterol/PEG-DMG2k = |
| | 35/16/46.5/2.5 |
| MD1/PS | MD1/DOPE/DOPS/Cholesterol/PEG-DMG2k = |
| | 35/16/5/46.5/2.5 |
| MD1/DDAB | MD1/DDAB/DOPE/Cholesterol/PEG-DMG2k = |
| | 35/40/16/46.5/2.5 |
| MD1/DDAB/PS | MD1/DDAB/DOPS/DOPE/Cholesterol/PEG-DMG = |
| | 35/40/5/16/46.5/2.5 |

wherein the above acronyms are defined as follows: 4-(dimethylamino)-butanoic acid, (10Z,13Z)-1-(9Z,12Z)-9,12-octadecadien-1-yl-10,13-nonadecadien-1-yl ester (MC3), 1,2-distearoyl-sn-glycero-3-phosphocholine (DSPC), PS (the same as DOPS), 1,2-dimyristoyl-rac-glycero-3-methoxypolyethylene glycol-2000 (DMG-PEG 2k), 3,6-bis [4-[bis (2-hydroxydodecyl)amino]butyl]-2,5-piperazinedione (MD1), dimethyldioctadecylammonium (bromide Salt) (DDAB), 1,2-dioleoyl-sn-glycero-3-phospho-L-serine (sodium salt) (DOPS), 1,2-dioleoyl-sn-glycero-3-phosphoethanolamine (DOPE).

### Example 2

### Enhanced In Vivo Delivery of MD1-LNPs

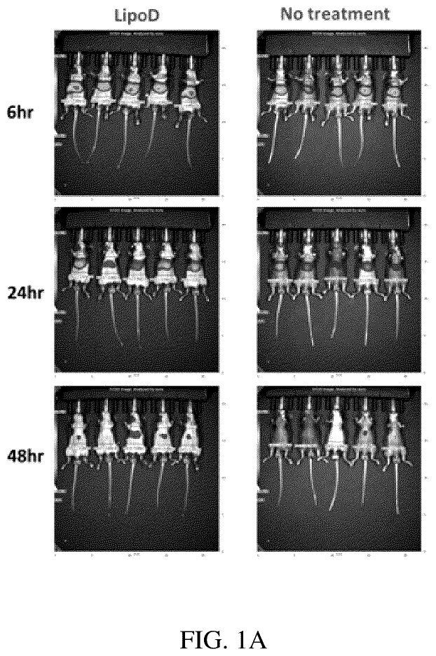

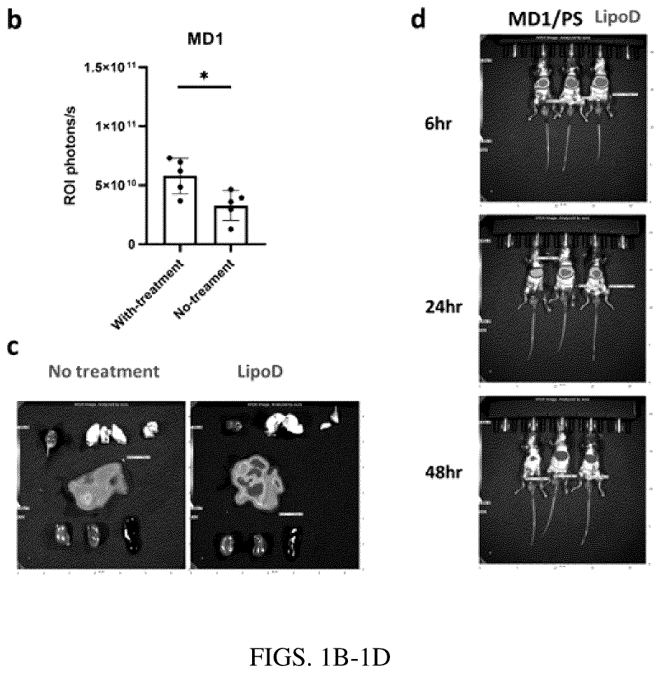

C57BL/6 mice at the age of 6-8 weeks were administered with MD1-LNP carrying Fluc-mRNA via retro-orbital injections at a dosage of 0.2 mg/kg. Bioluminescence imaging was performed at 6-h after transfection. Mice were injected with 150 mg/kg of D-luciferin diluted in PBS. Mice were pre-treated with liposome clodronate (LipoD, i.v. dose 18 h before LNP injection) to deplete macrophages and monocytes. Bioluminescence images were recorded by IVIS machine, and all images were analyzed using Aura Image Software. FIG. 1A shows mRNA delivery in LipoD pre-treated mice vs. no treatment mice at 6 h, 24 h, and 48 h. FIG. 1B shows results for with-treatment vs. no-treatment mice. FIG. 1C shows an exemplary no-treatment vs. LipoD pre-treatment. FIG. 1D shows LipoD pre-treatment mice that were administered MD1/PS LNPs. As shown in the foregoing figures, all LipoD treated mice showed significant higher bioluminescence signals in liver compared non-treated mice.

### Example 3

### Enhanced In Vivo Delivery of MD1/DDab-LNPs

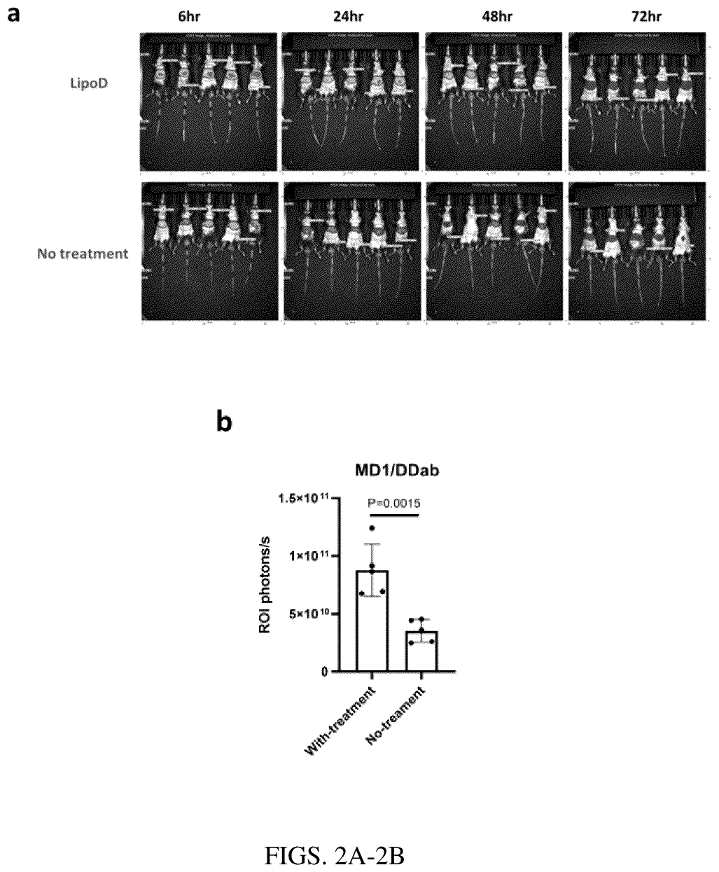

C57BL/6 mice at the age of 6-8 weeks were administered with MD1/DDab-LNPs carrying Fluc-mRNA via retro-orbital injections at a dosage of 0.2 mg/kg. Bioluminescence imaging was performed at 6-h after transfection. Mice were injected with 150 mg/kg of D-luciferin diluted in PBS. Mice were pre-treated with liposome clodronate (LipoD, intravenous dose 18 h before LNP injection) to deplete macrophages and monocytes. Bioluminescence images were recorded by IVIS machine, and all images were analyzed using Aura Image Software. FIG. 2A shows mRNA delivery in LipoD pre-treated mice vs. no treatment mice at 6 h, 24 h, 48 h, 72 h. FIG. 2B shows with treatment vs. no treatment mice. As shown in the foregoing figures, all LipoD treated mice showed significant higher bioluminescence signals in liver compared non-treated mice.

### Example 4

### Enhanced In Vivo Delivery of MD1/DDab/PS-LNPs

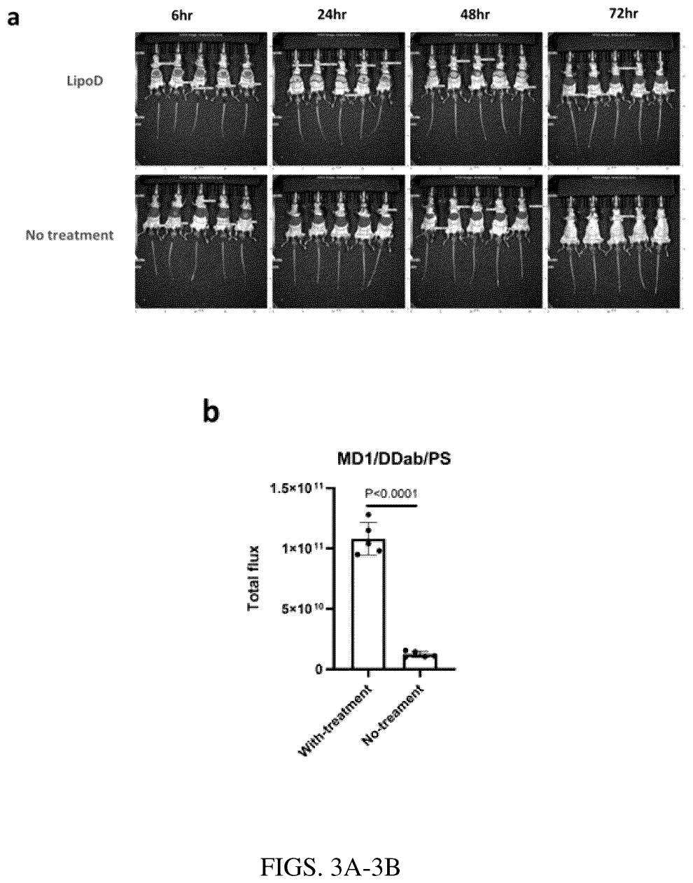

C57BL/6 mice at the age of 6-8 weeks were administered with MD1/DDab/PS-LNP carrying Fluc-mRNA via retro-orbital injections at a dosage of 0.2 mg/kg. Bioluminescence imaging was performed at 6-h after transfection. Mice were injected with 150 mg/kg of D-luciferin diluted in PBS. Mice were pre-treated with liposome clodronate (LipoD, intravenous dose 18 h before LNP injection) to deplete macrophages and monocytes. Bioluminescence images were recorded by IVIS machine, and all images were analyzed using Aura Image Software. FIG. 3A shows mRNA delivery in LipoD pre-treated mice vs. no treatment mice at 6 h, 24 h, 48 h, 72 h. FIG. 3B shows with treatment vs. no treatment mice. As shown in the foregoing figures, all LipoD treated mice showed 10-fold of bioluminescence signals compared those mice without LipoD treatment. Incorporation of DOPS to the formulation MD1/DDAB/LNPs reduces the expression of Fluc in non-treated mice, but demonstrates enhanced expression of Fluc in mice treated with LipoD.

### Example 5

### Enhanced In Vivo Expression of Secreting Protein

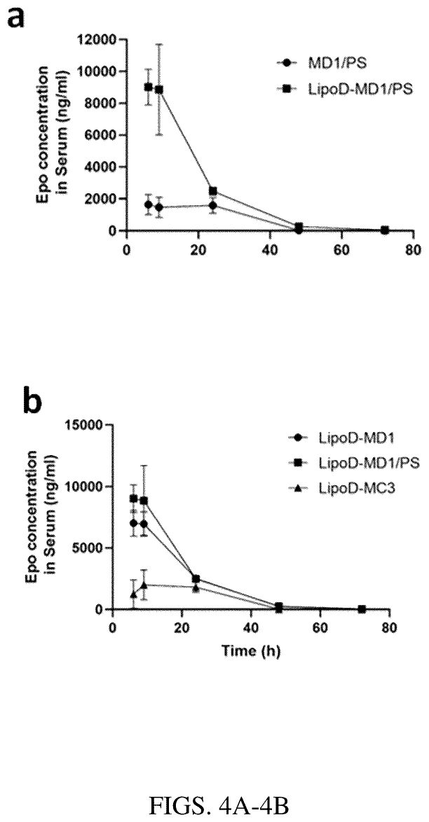

C57BL/6 mice at the age of 6-8 weeks were administered with hEPO-mRNA loaded LNPs via intravenous injections at a dosage of 0.2 mg/kg. Serum hEPO concentrations at 6 h, 9 h, 24 h, 48 h and 72 h post-injection were measured by Human EPO ELISA kit (Thermofisher). Mice were pre-treated with liposome clodronate (LipoD, IV injected at 18 h before LNP injection) to deplete macrophages and monocytes. The pharmacokinetic (PK) profile of hEPO expression after mRNA delivery by MD1/PS-LNPs is shown in FIG. 4A. LipoD treatment significantly improved the pharmacokinetics (PK) profile in both maximum concentration and area under curve, indicating an enhanced mRNA delivery after macrophage depletion. Similar results were observed in other LNP formulations (FIG. 4B). Therefore, the incorporation of PS lipids also enhanced the EPO expression in macrophage depleted mice compared to formulations without PS lipids.

### Example 6

### Screening Drugs That Could Enhance In Vivo mRNA Delivery

In this example, various small molecules or monoclonal antibodies with reported macrophage depletion capabilities were screened to evaluate the effectiveness to enhance mRNA delivery. Drugs used in this study include clodronate liposome (Lipo-Clodronate), anti-CD115, anti-CCR2, anti-Ly6C, anti-Gr-1 and zoledronate (10 μg, 50 μg and 100 μg).

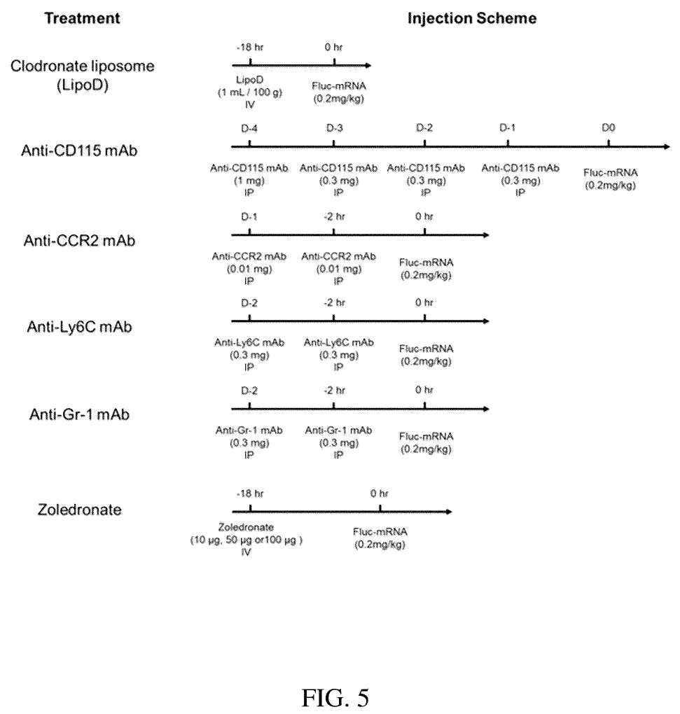

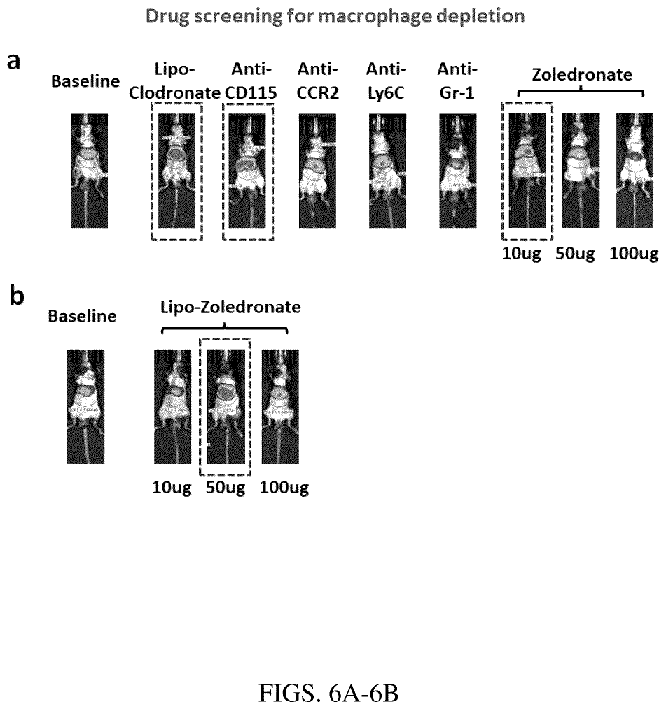

FIG. 5 lists treatment methods studied in this example. As shown in FIG. 6A, these drugs enhanced mRNA delivery to different degrees, particularly in the mouse treated with clodronate liposome, anti-cd115, Anti-CCR2, Anti-Ly6C and 10 μg of zoledronate. In contrast, Gr-1 and high dose of zoledronate decreased the Fluc expression. In a different experiment, zoledronate was encapsulated in liposomes (formulated with DSPC and cholesterol) and delivered to the mouse at different dosage. As shown in FIG. 6B, Enhanced Fluc expression was observed in dose-dependent manner.

### Example 7

### Enhanced In Vivo Gene Recombination Induced by Cre mRNA Delivery

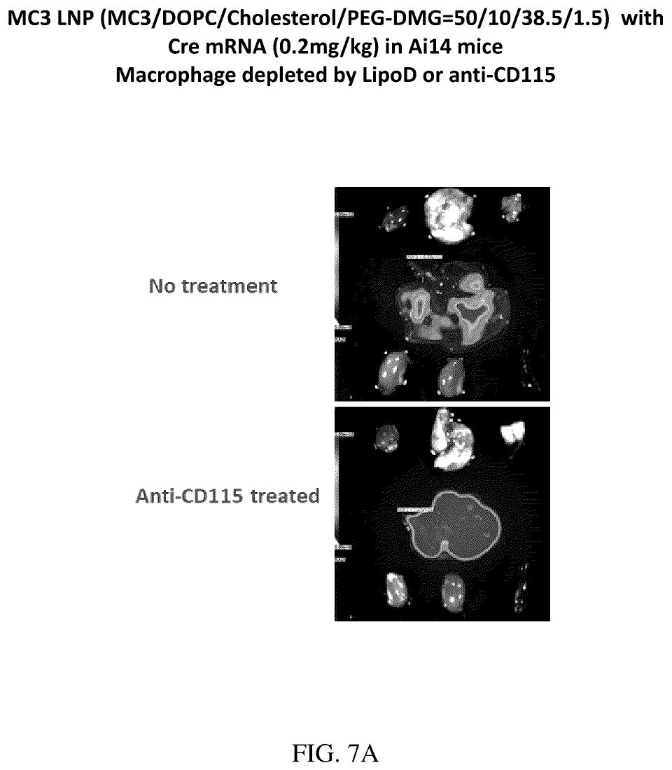

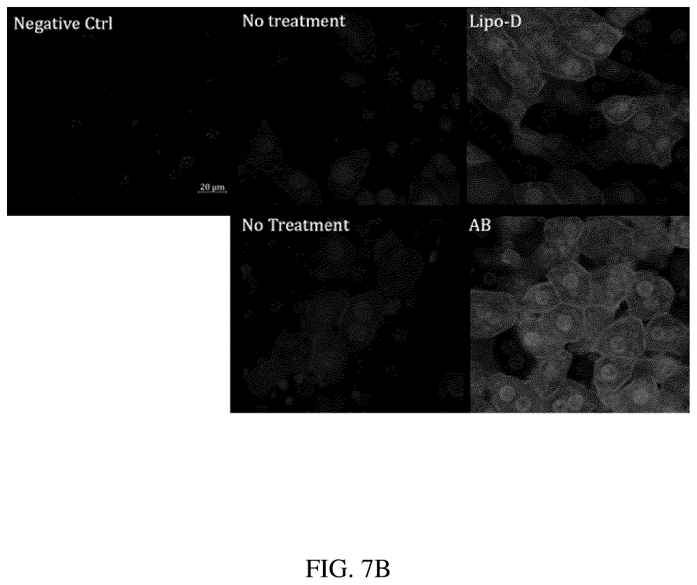

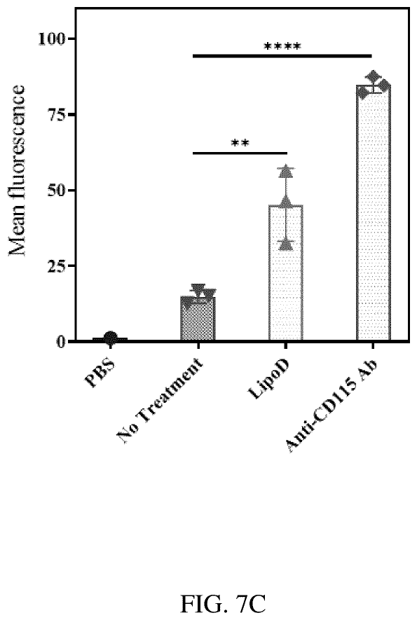

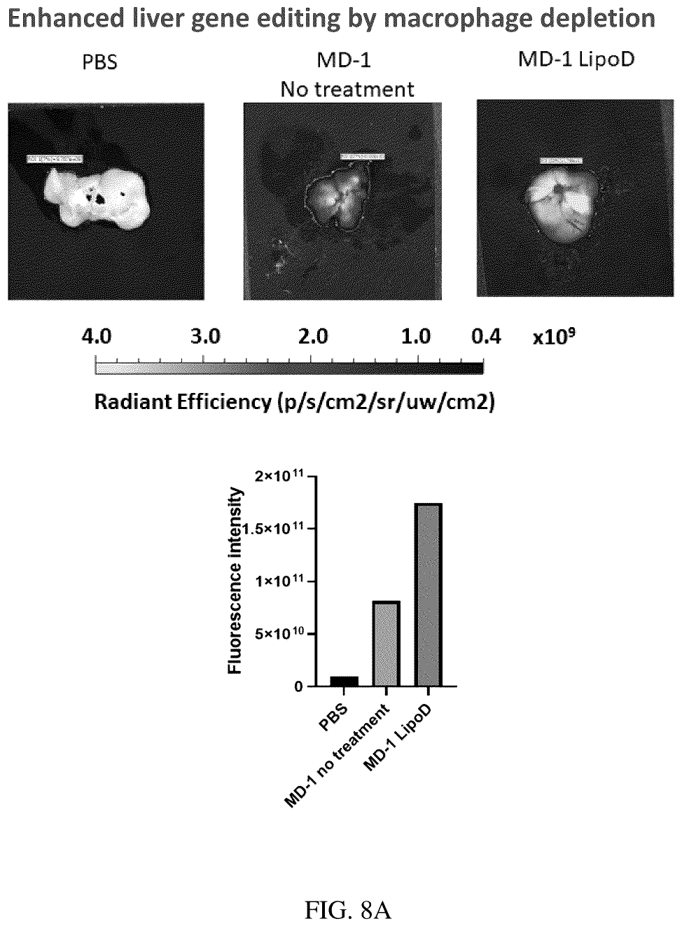

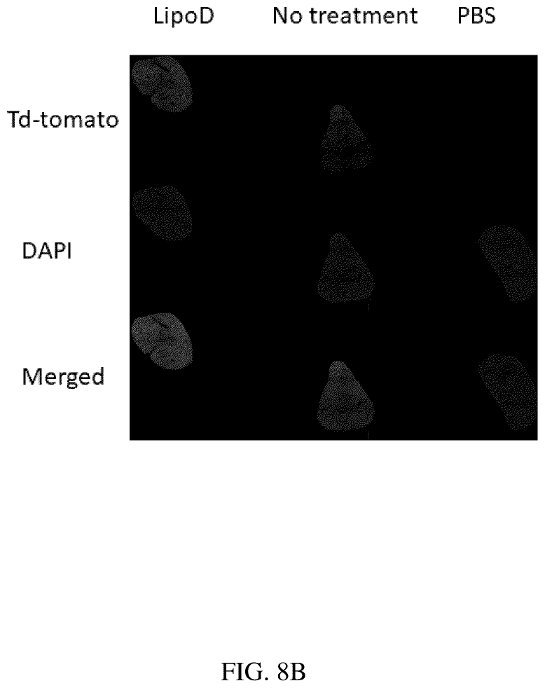

Ai14 mice at the age of 6-8 weeks were administered with Cre-mRNA loaded MC3-LNPs via retro-orbital injections at a dosage of 0.2 mg/kg. After 48 hours, mice were sacrificed and main organs (heart, lungs, lymph nodes, liver, spleen, kidneys) were harvested and imaged by IVIS machine. Macrophage depletion was achieved by pre-treating Ai14 mice with anti-CD115 intraperitoneally or LipoD intravenously, following a same injection schedule described in example 6. As shown in FIGS. 7A-7C, with treatment of anti-CD115, MC3-LNP encoding Cre protein triggered much stronger Cre mediated gene recombination in Ai14, resulting in stronger Td-tomato expression. Images of liver sections were further compared, and results are shown in FIGS. 8A-8B. No Td-tomato expression was found in control groups that were only received with PBS. MC3-LNPs successfully delivered Cre mRNA (0.2 mg/kg) in Ai-14 mice (no treatment groups), and part of liver cells was Td-tomato positive. “No treatment” means no pretreatment for depleting monocytes and/or macrophages. Macrophage depletion prior to LNP injection significantly enhanced the Cre mRNA delivery compared to no treatment groups. The Td-tomato expression was even higher in anti-CD115 treated Ai14 mice, suggesting a more radical way for macrophage depletion.

### Example 8

### Enhanced In Vivo Gene Editing Using Co-Delivery of Cas9-Encoding mRNA and sgRNA by MC3-LNPs

Ai14 mice at the age of 6-8 weeks were administered with MD-1 LNPs encapsulating Cas9 mRNA and sgRNA via retro-orbital injections at a dosage of 1.25 mg/kg (total RNA, mRNA/sgRNA=4/1, wt/wt). For enhanced mRNA delivery group, mice will be pre-treated with lipoD intravenously to deplete circulating and residential macrophages (liver). Mice without pre-treatment will be used as a control group. After 10 days, mice were sacrificed and livers were harvested and imaged by IVIS machine. Tissue sections were also prepared, fixed, stained and imaged using a confocal microscopy (Zeiss). Enhanced Td-tomato expression (FIGS. 8A-8B) was observed in macrophage depleted groups compared to control groups.

While there have been shown and described what are at present considered the preferred embodiments of the invention, those skilled in the art may make various changes and modifications which remain within the scope of the invention defined by the appended claims.

What is claimed is:

1. A method for delivery of a nucleic acid, comprising administering a lipid nanoparticle composition loaded with the nucleic acid to a subject in which monocytes and/or macrophages have been depleted, thereby delivering the nucleic acid into the subject.

2. A method for delivery of a nucleic acid into a subject, the method comprising:
a) depleting monocytes and/or macrophages in the subject; and
b) administering a lipid nanoparticle composition loaded with the nucleic acid to the subject, thereby delivering the nucleic acid into the subject.

3. The method of claim 2, wherein said depleting monocytes and/or macrophages is accomplished by administering small molecule drugs.

4. The method of claim 2, wherein said depleting monocytes and/or macrophages is accomplished by administering liposome compositions loaded with small molecule drugs.

5. The method of claim 2, wherein said depleting monocytes and/or macrophages is accomplished by administering antibodies configured to deplete monocytes and/or macrophages, wherein the antibodies include, but are not limited to anti-CD115, anti-CCR2, anti-Ly6C, anti-Gr-1.

6. The method of claim 2, wherein said depleting monocytes and/or macrophages is accomplished by administering one or more of liposome clodronate (LipoD) and liposome zoledronate.

7. The method of claim 2, wherein said depleting monocytes and/or macrophages is accomplished within hours, days, or weeks.

8. The method according to any one of claims 1-7, wherein the subject is a mammal.

9. The method of claim 8, wherein the mammal is selected from the group consisting of primates, humans, mice, rats, dogs, cats, rabbits, horses, sheep, and pigs.

10. The method according to any one of claims 1-9, wherein the nucleic acid encodes a protein that is expressed in the subject.

11. The method according to any one of claims 1-9, wherein the nucleic acid is a component of a gene editing machinery.

12. The method according to any one of claims 1-9, wherein the nucleic acid is delivered into one or more of liver, brain, spleen, lymph nodes, kidneys, and lungs.

13. The method according to any one of claims 1-9, wherein the nucleic acid comprises one or more selected from the group consisting of message RNA (mRNA), small interfering RNA (siRNA), microRNA (miRNA), circular RNA (circRNA), long-noncoding RNA (IncRNA), antisense oligonucleotide (ASO), CRISPR-related RNA, Cas nuclease mRNA, guide RNA, and single-guide RNA.

14. The method according to any one of claims 1-9, wherein the nucleic acid comprises an mRNA and the lipid nanoparticle composition further comprises targeting ligands to further enhance mRNA delivery.

15. The method of claim 14, wherein targeting ligands comprise phosphoserine (PS) moiety.

16. The method according to any one of claims 1-15, wherein the lipid nanoparticle composition comprises:
(i) at least one zwitterionic polymer-containing lipid in which a lipid moiety is covalently attached to a zwitterionic polymer;
(ii) at least one non-cationic lipid selected from charged and uncharged lipids, wherein the non-cationic lipid is not attached to a polymer;
(iii) at least one cationic or ionizable lipid containing a secondary, tertiary, or quaternary amino group; and
(iv) at least one nucleic acid substance.

17. The method of claim 16, wherein said lipid moiety in component (i) is a diacylglyceride.

18. The method according to any one of claims 16-17, wherein component (i) excludes a polyalkylene oxide segment.

19. The method according to any one of claims 16-18, wherein the zwitterionic polymer in component (i) is selected from the group consisting of a poly (carboxybetaine) (PCB), a poly(sulfobetaine), a poly(phosphobetaine), poly(phosphatidylcholine), glutamic acid-lysine (EK)-containing polypeptide, a poly(trimethylamine N-oxide) polymer and a poly(zwitterionic phosphatidyl serine).

20. The method according to any one of claims 16-18, wherein the zwitterionic polymer in component (i) is a betaine polymer.

21. The method of claim 20, wherein the betaine polymer is a poly (carboxybetaine), poly(sulfobetaine), or poly(phosphobetaine) polymer.

22. The method according to any one of claims 16-21, wherein the non-cationic lipid in component (ii) contains a zwitterionic moiety.

23. The method of claim 22, wherein the zwitterionic moiety is selected from the group consisting of a phosphobetaine, phosphatidylcholine, carboxybetaine, sulfobetaine, trimethylamine N-oxide, glutamic acid-lysine (EK)-containing peptide, and zwitterionic phosphatidyl serine moiety.

24. The method according to any one of claims 16-23, wherein the non-cationic lipid is selected from the group consisting of a dioleoylphosphatidylglycerol (DOPG), dipalmitoylphosphatidylglycerol (DPPG), dioleoylphosphatidylethanolamine (DOPE), palmitoyloleoyl-phosphatidylethanolamine (POPE), dipalmitoylphosphatidylethanolamine (DPPE), dimyristoylphosphoethanolamine (DMPE), distearoylphosphatidylethanolamine (DSPE), 16-O-monomethyl-phospho ethanolamine, 16-O-dimethyl-phosphoethanolamine, 18-1-trans-phosphoethanolamine, 1-stearoyl-2-oleoyl phosphatidyethanolamine (SOPE), and 1,2-dioleoyl-sn glycero-3-phophoethanolamine (transDOPE).

25. The method according to any one of claims 16-24, wherein component (ii) excludes a polyalkylene oxide segment.

26. The method according to any one of claims 16-25, wherein the non-cationic lipid in component (ii) is a phospholipid.

27. The method of claim 26, wherein the phospholipid is a phosphatidyl serine lipid.

28. The method according to any one of claims 16-27, wherein the cationic or ionizable lipid in component (iii) possesses a secondary, tertiary, or quaternary amino group.

29. The method according to any one of claims 16-27, wherein the cationic or ionizable lipid in component (iii) possesses a secondary, tertiary, or quaternary group along with a functional group which is negatively charged under physiological conditions.

30. The method according to any one of claims 16-27, where the cationic or ionizable lipid comprises: 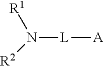
wherein:
R¹ and R² are independently selected from H and alkyl groups and wherein the alkyl group may be saturated, unsaturated, branched, and/or unbranched, and may optionally include one or more heteroatoms selected from N, O, F, Si, P, S, Cl, Br, and F;
L comprises a covalent linker group between N and A, wherein the covalent linker group is a linear or branched alkyl group containing 1-20 carbon atoms and may optionally include one or more heteroatoms selected from N, O, F, Si, P, S, Cl, Br, and F; and
A is a functional group that is negatively charged or capable of being deprotonated to form a negatively charged group at a pH of 5-8.

31. The method according to any one of claims 16-27, where the cationic or ionizable lipid is selected from the group consisting of: 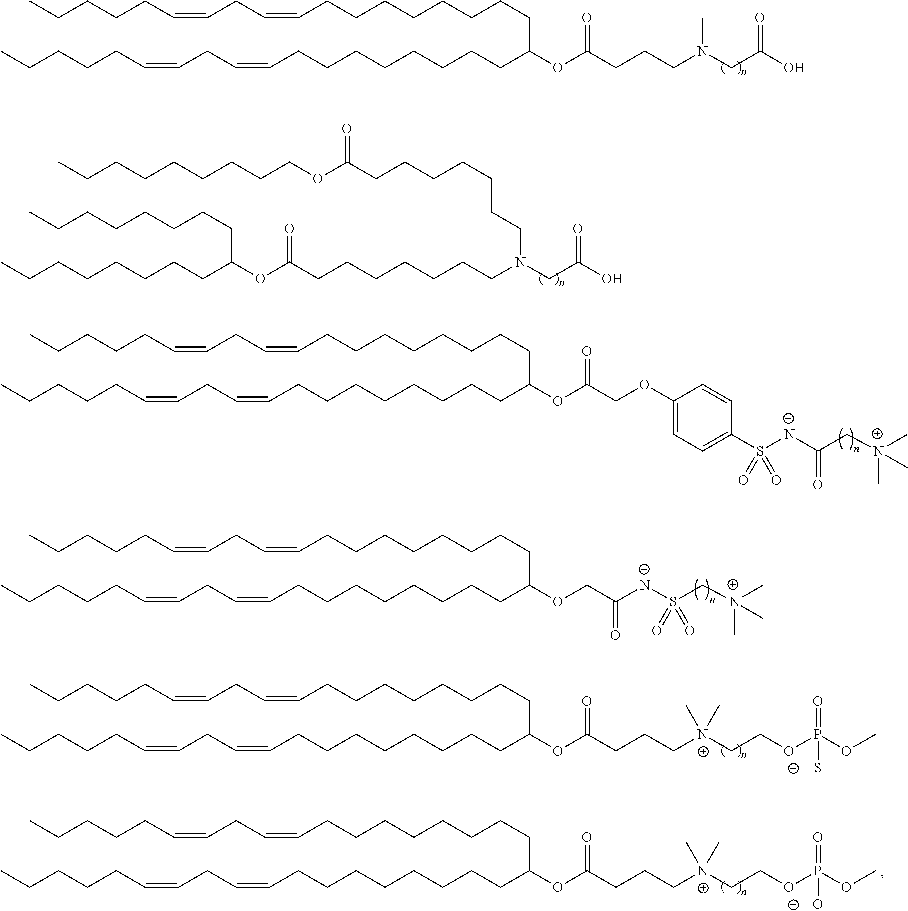
and
wherein n=1 to 10.

32. The method according to any one of claims 16-31, wherein the cationic or ionizable lipid in component (iii) excludes a polyalkylene oxide segment.

33. The method according to any one of claims 16-32, wherein the lipid nanoparticle composition further comprises: (v) cholesterol or a derivative thereof.

34. The method according to any one of claims 16-33, wherein the lipid nanoparticle composition comprises a lipid moiety attached to a secondary, tertiary, or quaternary amine group along with a functional group, wherein the functional group is negatively charged under physiological conditions.
